(19) United States
(12) Patent Application Publication
(10) Pub. No.: US 2026/0115122 A1
(43) Pub. Date: Apr. 30, 2026

(54) PERSONAL CARE COMPOSITIONS COMPRISING PANCRATIUM MARITIMUM

(71) Applicant: GALDERMA HOLDING SA, ZUG (CH)

(72) Inventors: Trong Dat NGO, Laval (CA); Claudia Victoria PEDRAZA NOCUA, Montreal (CA); Debbie GO, Singapore (SG)

(21) Appl. No.: 19/003,345

(22) Filed: Dec. 27, 2024

Related U.S. Application Data
(63) Continuation of application No. PCT/IB2022/056007, filed on Jun. 28, 2022.

(51) Int. Cl. A61K 8/67; A61Q 19/02

(52) U.S. Cl. CPC A61K 8/676; A61Q 19/02

(57) ABSTRACT

This disclosure provides personal care compounds that reduce dark spots, even skin tone, brighten skin, and prevent hyperpigmentation. This disclosure also provides methods of preventing hyperpigmentation in a subject using the personal care compositions described herein.

### CROSS-REFERENCE

This application is a continuation of International Patent Application No. PCT/IB2022/056007 filed Jun. 28, 2022, which application is hereby incorporated by reference in its entirety.

### FIELD OF DISCLOSURE

The present disclosure relates generally to the field of skin care. This disclosure provides personal care compounds that reduce dark spots, even skin tone, brighten skin, and prevent hyperpigmentation. This disclosure also provides methods of preventing hyperpigmentation in a subject using the personal care compositions described herein.

### BACKGROUND

Skin is the first barrier against environmental challenges including artificial blue light, particulate matter and ultraviolet (UV) light. Environmental challenges damage skin over time and accelerate skin aging.

While natural blue light provides benefits for the regulation of certain vital physiological mechanisms, overexposure to artificial blue light has a negative impact on photoreceptors on skin, damaging them. Therefore, artificial blue light accelerates skin aging. Wrinkles and hyperpigmentation on the skin are the most visible consequences of artificial blue light exposure.

Particulate matter, also known as particle pollution, is a mixture of extremely small particles and liquid droplets. Exposure to fine particles, which are 2.5 micrometers or smaller in diameter, can have a direct affect on keratinocytes and melanocytes, leading to gene expression changes relevant to skin aging, pigmentation, and inflammation.

Even though UV light is needed for Vitamin D production, it also has long been linked to skin damage. Excessive UV light exposure can also cause DNA damage, and may lead to skin cancer.

Sensitive skin has a disrupted skin barrier which affects the skin texture which in turn reflects less light. This is often perceived as dull and unhealthy-looking skin. Sensitive skin is more prone to inflammation which in turns has been shown to trigger production of melanin pigments or melanogenesis. Sensitive skin is more reactive to external stress such as UV exposure, pollution and oxidation; therefore, more prone to uneven skin and hyperpigmentation as a result.

Compositions and methods to protect the skin from environmental challenges and their effects on aging are highly needed.

### SUMMARY OF THE DISCLOSURE

An aspect of the disclosure is directed to a personal care composition comprising, by weight: (a) 0.1%-10% niacinamide, (b) 0.1%-10% extract of sea daffodil (Pancratium maritimum), (c) 0.1%-10% acetyl glycyl β-alanine, and (d) 0.1%-5% Vitamin C derivative.

In some embodiments, the Vitamin C derivative is selected from ethyl ascorbic acid, ascorbyl glucoside (AA2G), ascorbic acid, ascorbyl palmitate or magnesium ascorbyl phosphate (MAP). In some embodiments, the Vitamin C derivative is ethyl ascorbic acid.

In some embodiments, the composition comprises 0.1%-2% Vitamin C derivative by weight. In some embodiments, the composition comprises 0.1%-1% Vitamin C derivative by weight.

In some embodiments, the personal care composition further comprises at least one of the following: water, humectant, pigment, antioxidant, preservative, gelling agent, emulsifier, solvent, pH balancing agent, and emollient.

In some embodiments, the humectant comprises at least one of xylitylglucoside, anhydroxylitol, and xylitol.

In some embodiments, the pigment comprises at least one of zinc oxide, titanium dioxide and iron oxide.

In some embodiments, the personal care composition further comprises at least one of the following: hydrolyzed hyaluronic acid, glycerin, sodium hydroxide, phenoxyethanol, xantham gum, sodium polyacryloyldimethyl taurate, undecane, tridecane, tocopherol, dimethicone, titanium dioxide, tin oxide, and calcium aluminum borosilicate.

In some embodiments, the personal care composition is a lotion, a gel, a mask, or a spray.

Another aspect method for preventing or reducing hyperpigmentation in a subject, comprising administering the subject a personal care composition comprising a skin lightening mixture, and an antioxidant, wherein the skin lightening mixture comprises niacinamide, extract of sea daffodil (Pancratium maritimum), and acetyl Glycyl β-Alanine, and wherein the antioxidant comprises a Vitamin C derivative.

In some embodiments, the personal care composition comprises, by weight: (a) 0.1%-10% niacinamide, (b) 0.1%-10% extract of sea daffodil (Pancratium maritimum), (c) 0.1%-10% acetyl Glycyl β-Alanine (also known as GenoWhite™), and (d) 0.1%-5% Vitamin C derivative.

In some embodiments, the Vitamin C derivative is ethyl ascorbic acid. In some embodiments, the Vitamin C derivative is selected from ethyl ascorbic acid, ascorbyl glucoside (AA2G), ascorbic acid, ascorbyl palmitate or magnesium ascorbyl phosphate (MAP).

In some embodiments, the composition comprises 0.1%-2% Vitamin C derivative by weight. In some embodiments, the composition comprises 0.1%-1% Vitamin C derivative by weight.

In some embodiments, the personal care composition further comprises at least one of the following: water, humectant, pigment, antioxidant, preservative, gelling agent, emulsifier, solvent, pH balancing agent, and emollient.

In some embodiments, the humectant comprises at least one of xylitylglucoside, anhydroxylitol, and xylitol.

In some embodiments, the pigment comprises at least one of zinc oxide, titanium dioxide and iron oxide.

In some embodiments, the personal care composition further comprises at least one of the following: hydrolyzed hyaluronic acid, glycerin, sodium hydroxide, phenoxyethanol, xantham gum, sodium polyacryloyldimethyl taurate, undecane, tridecane, tocopherol, dimethicone, titanium dioxide, tin oxide, and calcium aluminum borosilicate.

In some embodiments, the personal care composition is a lotion, a gel, a mask, or a spray.

In some embodiments, the composition is administered topically.

In some embodiments, the subject suffers from sensitive skin.

### BRIEF DESCRIPTION OF THE DRAWINGS

FIG. 1 shows the result of an ROS production assay. ROS production after 24 h of contact with 6 test formulations and reference Ascorbic acid is showed. Mean of data obtained from 3 cultures for each condition±standards deviations are reported to the untreated/non-irradiated control condition. Statistical analysis was performed as compared to untreated irradiated control with values of 0.01<p<0.05 considered as significant (*) and p<0.001 as very highly significant (***).

FIG. 2 shows the results of a melanin reduction assay. The histograms represent values obtained from 3 replicates (n=3) for each condition. A statistical analysis (Student-t-test) was performed as compared to untreated control with 0.01<p-values<0.05 considered as significant (*), 0.001<p-values<0.01 as highly significant (**) and p-values<0.001 as very highly significant (***).

FIG. 3 shows the results of a tyrosinase inhibition assay. The histograms represent values obtained from 3 replicates (n=3) for each condition. A statistical analysis (Student-t-test) was performed as compared to untreated control with 0.01<p-values<0.05 considered as significant (*), 0.001<p-values<0.01 as highly significant (**) and p-values<0.001 as very highly significant (***).

### DETAILED DESCRIPTION

### Definitions

All percentages and ratios used herein are by weight of the total composition, unless otherwise designated. All measurements are understood to be made at ambient conditions, where “ambient conditions” means conditions at about 25° C., under about one atmosphere of pressure, and at about 50% relative humidity, unless otherwise designated. All numeric ranges are inclusive of narrower ranges; delineated upper and lower range limits are combinable to create further ranges not explicitly delineated.

The compositions of the present disclosure can comprise, consist essentially of, or consist of, the components described herein. As used herein, “consisting essentially of” means that the composition or component may include additional ingredients, but only if the additional ingredients do not materially alter the basic and novel characteristics of the claimed compositions or methods.

As used herein, the term about refers to ±10% of a given value.

The term “apply” or “application,” as used in reference to a composition, means to apply or spread the compositions of the present disclosure onto keratinous tissue such as the epidermis.

The term “derivatives” includes an ester, ether, amide, hydroxy, and/or salt structural analogue of a compound of interest.

The term “dermatologically acceptable” means that the compositions or components described are suitable for use in contact with human skin tissue without undue toxicity, incompatibility, instability, allergic response, and the like.

The term “hyperpigmentation” refers to areas of skin where the pigmentation is greater than that of adjacent skin areas (for example, pigmented spots, age spots, moles, and the like). The hyperpigmentation of the present invention includes (but is not limited to) regional hyperpigmentation caused by excessive melanocyte activity, other local hyperpigmentation caused by benign melanocyte hyperactivity and proliferation, disease-related hyperpigmentation, and excessive occasional pigmentation, such as those hyperpigmented due to photosensitivity, genetic makeup, chemical uptake or other exposure (e.g., UV exposure), age, and scar formation after lesions. Skin pigmentation can be assessed in a number of ways, including, but not limited to, visual assessments using, for example, the von Luschan chromatic scale, the Fitzpatrick skin typing test (Fitzpatrick et al., Archives of Dermatology, vol. 124.6 (1988): pp. 869-871, 1988) and the Taylor Hyperpigmentation Scale (Taylor et al., Cutis, vol. 76, No. 4, pp. 270-274, 2005) and reflectance spectrophotometry methods (Zonios, et al., J. Invest. Dermatol, vol. 117, pp. 1452-1457, 2001). For example, the Fitzpatrick skin typing test includes six types of skin (I-VI), and Type VI skin that becomes Type V or less has been “brightened” as the term is used herein.

The term “keratinous tissue” refers to keratin-containing layers disposed as the outermost protective covering of mammals (e.g., humans, dogs, cats, etc.) which includes, but is not limited to, skin, lips, hair, toenails, fingernails, cuticles, hooves, etc.

As used herein, the term “pH balancing” agent refers to any compound (e.g., an acid or a base) useful for adjusting the pH of a composition to a specific value (e.g., 7, 8, 9, etc) or a specific range (e.g., pH=about 5 to about 7, about 7 to about 9, and the like). In some embodiments, the pH balancing agent is added to the composition in an amount effective to adjust the pH level of the composition to a pH greater than about pH 3 and less than about pH 9. Generally, pH of about 4 to about pH of about 6.5 is permissible. Additionally, pH of about 4.3 to about 5.4 is also permissible. The acceptable pH for the formulation is about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, and about 6.0, about 6.1, about 6.2, about 6.3, about 6.4 about 6.5, about 6.6, about 6.7, about, 6.8, about 6.9, and about 7.0. In some embodiments, the pH balancing agent is added to the composition in an amount effective to adjust the pH level of the composition to about 4 to about 7.

Exemplary pH balancing agents include, but are not limited to, ammonium aluminum sulfate, ammonium bicarbonate, ammonium carbonate, ammonium citrate dibasic, ammonium citrate monobasic, ammonium hydroxide, ammonium phosphate dibasic, ammonium phosphate monobasic, calcium acetate, calcium acid pyrophosphate, calcium carbonate, calcium chloride, calcium citrate, calcium fumarate, calcium Gluconate, calcium Hydroxide, calcium Lactate, calcium oxide, calcium phosphate dibasic, calcium phosphate monobasic, calcium phosphate tribasic, calcium sulfate, cream of tartar, glucono-delta-lactone, magnesium carbonate, magnesium citrate, magnesium fumarate, magnesium hydroxide, magnesium oxide, magnesium phosphate, magnesium sulfate, manganese sulfate, potassium acid tartrate, potassium aluminum sulfate, potassium bicarbonate, potassium carbonate, potassium chloride, potassium citrate, potassium fumarate, potassium hydroxide, potassium lactate, potassium phosphate dibasic, potassium phosphate tribasic, potassium sulfate, potassium tartrate, potassium tripolyphosphate, sodium acetate, sodium acid pyrophosphate, sodium acid tartrate, sodium aluminum phosphate, sodium aluminum sulfate, sodium bicarbonate, sodium bisulphate, sodium carbonate, sodium citrate, sodium fumarate, sodium gluconate, sodium hexametaphosphate, sodium hydroxide, sodium lactate, sodium phosphate dibasic, sodium phosphate monobasic, sodium phosphate tribasic, sodium potassium hexametaphosphate, sodium potassium tartrate, sodium potassium tripolyphosphate, sodium pyrophosphate tetrabasic, and sodium tripolyphosphate.

The term “safe and effective amount” means an amount of a compound or composition sufficient to significantly induce a positive benefit.

The term “sensitive skin” refers to a neurosensory skin condition that results in disparate clinical signs, including irritation, erythema or dry skin. More precisely, sensitive skin is a syndrome defined by the occurrence of unpleasant sensations, such as stings, burns, pain, pruritus or tingling, in response to stimuli that should not normally cause such sensations; in addition, sensitive skin is characterized by the absence of a link between these sensations and any skin pathology (Misery et al., Acta Derm Venereol 2017, 97:4-6). Suitable assays for measuring sensitivity, inflammation, irritation of the skin are known in the art. One such assay is the Jordan-King assay, as set forth in Jordan, W. P. 1994, Jordan/King modification of the Draize Repeat Insult Patch Test, Clairol Study #94046, Test Dates Oct. 3, 1994-Nov. 11, 1994, the entire contents of which are hereby incorporated by reference.

The term “leave-on,” in reference to personal care compositions, means a composition intended to be applied to and allowed to remain on the keratinous tissue. These leave-on compositions are to be distinguished from compositions which are applied to the skin and subsequently (in a few minutes or less) removed either by washing, rinsing, wiping, or the like. Leave-on compositions exclude rinse-off applications such as shampoos, facial cleansers, hand cleansers, body wash, or body cleansers. The leave-on compositions may be substantially free of cleansing or detersive surfactants. For example, “leave-on compositions” may be left on the keratinous tissue for at least 15 minutes. For example, leave-on compositions may comprise less than 1% detersive surfactants, less than 0.5% detersive surfactants, or 0% detersive surfactants. The compositions may, however, contain emulsifying or other processing surfactants that are not intended to provide any significant cleansing benefits when applied topically to the skin.

The term “rinse-off” means the intended personal care composition usage includes application to the keratinous tissue followed by rinsing and/or wiping the product from the keratinous tissue within a few seconds to minutes of the application step. The product is generally applied and rinsed in the same usage event, for example, a shower.

The term “soluble” means at least about 0.1 g of solute dissolves in 100 ml of solvent, at 25° C. and 1 atm of pressure.

As used herein, the term “personal care composition” refers to compositions suitable for topical application on mammalian keratinous tissue.

### Personal Care Compositions

The instant disclosure provides personal care compositions that reduce dark spots, even skin tone, brighten skin, increase radiance and prevent hyperpigmentation. Without committing to one particular theory, the personal care compositions of the instant disclosure prevent hyperpigmentation of skin by inhibiting stimulation of melanin release (e.g., by inhibiting the activation of melanocytes and inhibiting melanin synthesis) and preventing melanin transfer to the epidermis. The personal care compositions of the instant disclosure are suitable for use on sensitive skin and shows dark spot reduction as early as within 14 days of daily use. The personal care compositions of the instant disclosure improve the skin's production of hyalauronic acid.

An aspect of the disclosure is directed to a personal care composition comprising, by weight: (a) 0.1%-10% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) niacinamide, (b) 0.1%-10% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) extract of sea daffodil (Pancratium maritimum), (c) 0.1%-10% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) acetyl glycyl β-alanine, and (d) 0.1%-5% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%) Vitamin C derivative.

Another aspect of the disclosure is directed to a personal care composition comprising, by weight: (a) 2%-8% (e.g., 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%) niacinamide, (b) 0.1%-5% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%) extract of sea daffodil (Pancratium maritimum), (c) 0.1%-5% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%) acetyl glycyl β-alanine, and (d) 0.1%-2% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%) Vitamin C derivative. In a specific embodiment the personal care composition comprises, by weight: (a) 3%-6% (e.g., 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%) niacinamide, (b) 0.5%-3% (e.g., 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%) extract of sea daffodil (Pancratium maritimum), (c) 0.5%-3% (e.g., 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%) acetyl glycyl β-alanine, and (d) 0.5%-1.5% (e.g., 0.5%, 0.6%, 0.7%, 0.8%, 0.9%, 1%, 1.1%, 1.2%, 1.3%, 1.4%, 1.5%) Vitamin C derivative. In a specific embodiment the personal care composition comprises, by weight: (a) 4%-5% (e.g., 4%, 4.1%, 4.2%, 4.3%, 4.4%, 4.5%, 4.6%, 4.7%, 4.8%, 4.9%, or 5%) niacinamide, (b) 1%-2% (e.g., 1%, 1.1%, 1.2%, 1.3%, 1.4%, 1.5%, 1.6%, 1.7%, 1.8%, 1.9%, or 2%) extract of sea daffodil (Pancratium maritimum), (c) 0.7%-1% (e.g., 0.7%, 0.8%, 0.9%, or 1%) acetyl glycyl β-alanine, and (d) 0.7%-1% (e.g., 0.7%, 0.8%, 0.9%, or 1%) Vitamin C derivative.

In some embodiments, the vitamin C derivative is selected from ascorbyl glucoside (AA-2G), ascorbic acid, ascorbyl palmitate or magnesium ascorbyl phosphate (MAP).

In some embodiments, the vitamin C derivative is ascorbyl palmitate having the formula:

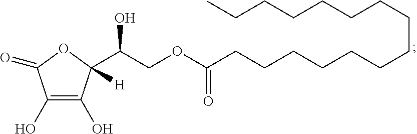 [SMILES: CCCCCCCCCCCCCCCC(=O)OC[C@H](O)[C@H]1OC(=O)C(O)=C1O]

derivatives thereof; and salts of any of the foregoing.

In some embodiments, the vitamin C derivative is magnesium ascorbyl phosphate having the formula:

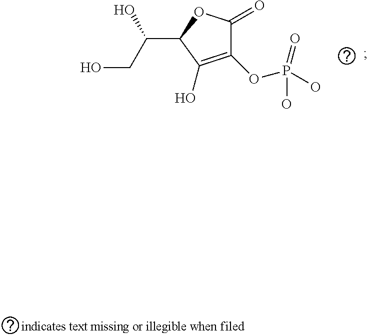 [SMILES: O=C1O[C@H]([C@@H](O)CO)C(O)=C1OP(=O)(O)O]

derivatives thereof; and salts of any of the foregoing.

In some embodiments, the vitamin C derivative is ethyl ascorbic acid having the formula:

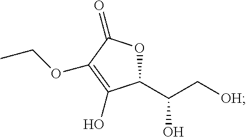

derivatives thereof; and salts of any of the foregoing.

In some embodiments, the vitamin C derivative is ascorbyl glucoside (AA-2G) having the formula:

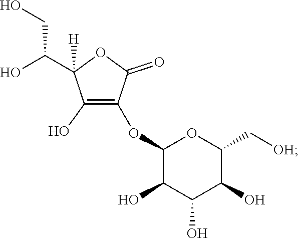 [SMILES: O=C1O[C@H]([C@H](O)CO)C(O)=C1O[C@H]1O[C@H](CO)[C@@H](O)[C@H](O)[C@H]1O]

derivatives thereof; and salts of any of the foregoing.

In some embodiments, the personal care composition comprises 0.1%-2% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%) Vitamin C derivative by weight. In some embodiments, the personal care composition comprises 0.1%-1% (e.g., 0.1%, 0.2%, 0.3%, 0.4%, 0.5%, 0.6%, 0.7%, 0.8%, 0.9%, 1%) Vitamin C derivative by weight.

In some embodiments, the personal care composition further comprises at least one of the following: water, humectant, pigment, antioxidant, preservative, gelling agent, emulsifier, solvent, pH balancing agent, and emollient.

In some embodiments, the humectant comprises at least one of xylitylglucoside, anhydroxylitol, and xylitol. In some embodiments, the humectant comprises each of xylitylglucoside, anhydroxylitol, and xylitol. In some embodiments, the humectant makes up, by weight, 0.01-0.5% of the personal care composition.

In some embodiments, the pigment comprises at least one of zinc oxide, titanium dioxide and iron oxide.

In some embodiments, the personal care composition further comprises at least one of the following: hydrolyzed hyaluronic acid, glycerin, sodium hydroxide, phenoxyethanol, xantham gum, sodium polyacryloyldimethyl taurate, undecane, tridecane, tocopherol, dimethicone, titanium dioxide, tin oxide, and calcium aluminum borosilicate. In some embodiments, the personal care composition comprises each of the following: hydrolyzed hyaluronic acid, glycerin, sodium hydroxide, phenoxyethanol, xantham gum, sodium polyacryloyldimethyl taurate, undecane, tridecane, tocopherol, dimethicone, titanium dioxide, tin oxide, and calcium aluminum borosilicate.

In some embodiments, the personal care composition comprises at least one of the following: hydrolyzed hyaluronic acid (0.001%-0.05% by weight of the personal care composition), glycerin (0.1%-7% by weight), sodium hydroxide (0.001%-0.1% by weight of the personal care composition), phenoxyethanol (0.1%-1% by weight of the personal care composition), xantham gum (0.05%-0.5% by weight of the personal care composition), sodium polyacryloyldimethyl taurate (0.1%-1.2% by weight of the personal care composition), an emollient composition comprising undecane, tridecane, and tocopherol (0.05%-0.5% by weight of the personal care composition), dimethicone (0.5%-5% by weight), a pigment composition comprising titanium dioxide, tin oxide, and calcium aluminum borosilicate (0.005%-0.05% by weight of the personal care composition).

In some embodiments, the emollient composition comprises Cetiol Ultimate™ from BASF which comprises undecane, tridecane, and tocopherol. In some embodiments, the personal care composition comprises between 0.05% and 0.5% (e.g., 0.05%, 0.06%, 0.07%, 0.08%, 0.09%, 0.1%, 0.2%, 0.3%, 0.4%, or 0.5%) Cetiol Ultimate™.

In some embodiments, the pigment composition comprises Syncrystal Gold™ from Eckart which comprises titanium dioxide, tin oxide, and calcium aluminum borosilicate. In some embodiments, the personal care composition comprises between 0.005% and 0.05% (e.g., 0.005%, 0.01%, 0.02%, 0.03%, 0.04%, or 0.05%) Syncrystal Gold™.

In some embodiments, the personal care composition further comprises an exfoliator. In some embodiments, the exfoliator is jojoba beads containing vitamin E.

In some embodiments, the personal care composition is a lotion, a gel, a mask, or a spray. The personal care composition of the disclosure may be a skin care, anti-perspirant, deodorant, cosmetic, or hair care product. The personal care composition may be used as, for example, a moisturizer, conditioner, anti-aging compound, skin lightener, skin mask, sunscreen, sunless tanner, shave preparation, lipstick, foundation, mascara, after-shave, and combinations thereof. In certain embodiments, the composition is applied to the face, neck, hands, arms, and other typically exposed areas of the body.

The personal care composition may involve a wide variety of forms. Non-limiting examples include simple solutions (e.g., water or oil-based), dispersions, and emulsions. The personal care composition may be substantially anhydrous. “Substantially anhydrous” means that the composition comprises no more than about 1%, 0.5%, or, 0% water. The personal care compositions may be fluid or solid (gels, sticks, flowable solids, amorphous materials). In certain embodiments, the personal care composition is in the form of an emulsion. Emulsion may be generally classified as having a continuous aqueous phase (e.g., oil-in-water and water-in-oil-in-water) or a continuous oil phase (e.g., water-in-oil and oil-in-water-in-oil).

In some embodiments, the personal care composition is a leave-on composition.

In some embodiments, the personal care composition is a rinse-off composition.

In select embodiments, the personal care composition may be in a form comprising at least one discrete, visually distinct first phase and at least one discrete, visually distinct second phase. For purposes of these select embodiments, “visually distinct” means that the phases can be separately seen by the human eye as distinctly separate regions (i.e., not emulsions or dispersions of particles. In some embodiments, at least one phase forms a stable pattern, for example a continuous or discontinuous line, a spiral, a curve, or other geometric shape, within a transparent phase, where “within” means that one phase is substantially surrounded by the other phase the and does not contact the side of a container. Alternatively, the phases may form a swirled pattern, wherein both phases alternately contact the side of a container and wherein the width of each of phase, when viewed through the side of a transparent container, is substantially constant, but may differ from each other. Alternatively, the phases may form a marbled pattern, wherein the phases alternately contact the side of the container and wherein the width of the individual phases, when viewed through the side of a transparent container, may vary throughout the composition. In one embodiment, the first phase is a transparent, clear or translucent aqueous phase and the second phase is either an opaque white or colored non-aqueous phase. In another alternative embodiment, at least one aqueous phase forms a pattern within a non-aqueous phase. In some embodiments, the instant personal care compositions may comprise a three or more visually distinct and stable phases. Discrete, visually distinct multi-phase compositions are described in U.S. Patent Application Publication Nos. 2007/0297996, 2004/0057920, and 2004/0219119, which are all incorporated by reference in their entireties.

### Carriers

The personal care composition may comprise a carrier. Carriers may be selected for various stability, aesthetics, and/or compatibility with other materials present in the personal care composition.

Suitable carriers include water and/or water-soluble solvents. The personal care composition may comprise from about 19% to about 95% by weight of water and/or water-equivalent solvent. The composition may comprise from about 1%, 3%, 5%, 10%, 15%, 20%, 25%, 30%, 35%, 40%, 45%, 50%, 55%, 60%, 65%, 70%, 75%, 80%, 85%, or 90% to about 90%, 85%, 80%, 75%, 70%, 65%, 60%, 55%, 50%, 45%, 40%, 35%, 30%, 25%, 20%, 15%, 10%, or 5% water and/or a water-equivalent solvent. “Water-equivalent solvent” refers to a compound which has a similar ability as water to solubilize a material. Suitable water-equivalent solvents include monohydric alcohols, dihydric alcohols, polyhydric alcohols, glycerol, glycols, polyalkylene glycols such as polyethylene glycol, and mixtures thereof. Particularly suitable solvents, include lower aliphatic alcohols such as ethanol, propanol, butanol, isopropanol; diols such as 1,2-propanediol, 1,3-propanediol, butanediol, pentanediol, hexanediol, heptanediol, decanediol; glycerin; water, and mixtures thereof. In certain embodiments, the personal care composition comprises water, diols, glycerin, and combinations thereof.

Suitable carriers also include oils. The personal care composition may comprise from about 1% to about 95% by weight of one or more oils. The composition may comprise from about 1%, 3%, 5%, 10%, 15%, 20%, 25%, 30%, 35%, 40%, 45%, 50%, 55%, 60%, 65%, 70%, 75%, 80%, 85%, or 90% to about 90%, 85%, 80%, 75%, 70%, 65%, 60%, 55%, 50%, 45%, 40%, 35%, 30%, 25%, 20%, 15%, 10%, or 5% of one or more oils. Oils may be used to solubilize, disperse, or carry materials that are not suitable for water or water equivalent solvents. Suitable oils include silicones, hydrocarbons, esters, fatty amides, ethers, and mixtures thereof. Oils may be fluid at room temperature. However, certain personal care product forms (i.e., solid or semi-solid stick) may require non-fluid oils. The oils may be volatile or nonvolatile. “Non volatile” means a material that exhibits a vapor pressure of no more than about 0.2 mm Hg at 25° C. at one atmosphere and/or a material that has a boiling point at one atmosphere of at least about 300° C. “Volatile” means that the material exhibits a vapor pressure of at least about 0.2 mm, of mercury at 20° C. Volatile oils may be used to provide a lighter feel when a heavy, greasy film is undesirable.

Suitable oils include volatile oils. In certain embodiments, the volatile oils may have a viscosity ranging from about 0.5 to about 5 centistokes 25° C. Volatile oils may be used to promote more rapid drying of the skin care composition after it is applied to skin. Nonvolatile oils are also suitable for use in the composition. Nonvolatile oils are often used for emolliency and protective properties. Nonvolatile oils may have a viscosity ranging from about 5 to about 800,000 cst (or greater) or from about 20 to about 200.000 cst.

Suitable silicone oils include polysiloxanes. Polylsiloxanes may have a viscosity of from about 0.5 to about 1,000.000 centistokes at 25° C. Such polysiloxanes can be represented by the general chemical formula:

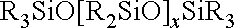

wherein R is independently selected from hydrogen or C₁₋₃₀ straight or branched chain, saturated or unsaturated alkyl, phenyl or aryl, trialkylsiloxy; and x is an integer from 0 to about 10,000, chosen to achieve the desired molecular. In certain embodiments, R is hydrogen, methyl, or ethyl. Commercially available polysiloxanes include the polydimethylsiloxanes, which are also known as dimethicones, examples of which include the DM-Fluid series from Shin-Etsu, the Vicasil® series sold by Momentive Performance Materials Inc., and the Dow Corning® 200 series sold by Dow Corning Corporation. Specific examples of suitable polydimethylsiloxanes include Dow Corning® 200 fluids (also sold as Xiameter® PMX-200 Silicone Fluids) having viscosities of 0.65, 1.5, 50, 100, 350, 10,000, 12,500 100,000, and 300,000 centistokes.

Suitable dimethicones include those represented by the chemical formula:

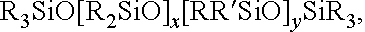

wherein R and R′ are each independently hydrogen or C₁₋₃₀ straight or branched chain, saturated or unsaturated alkyl, aryl, or trialkylsiloxy; and x and y are each integers of 1 to 1,000,000 selected to achieve the desired molecular weight. Suitable silicones include phenyl dimethicone (Botansil™ PD-151 from Botanigenies, Inc.), diphenyl dimethicone (KF-53 and KF-54 from Shin-Etsu), phenyl trimethicone (556 Cosmetic Grade Fluid from Dow Corning), or trimethylsiloxyphenyl dimethicone (PDM-20, PDM-200, or PDM-1000 from Wacker-Belsil). Other examples include alkyl dimethicones wherein at least R′ is a fatty alkyl (e.g., C₁₂₋₂₂). A suitable alkyl dimethicone is cetyl dimethicone, wherein R′ is a straight C16 chain and R is methyl. Cetyl dimethicone, is available as s 2502 Cosmetic Fluid from Dow Corning or as Abil Wax 9801 or 9814 from Evonik Goldschmidt GmbH.

Cyclic silicones are one type of silicone oil that may be used in the personal care compositions of the instant disclosure. Such silicones have the general formula:

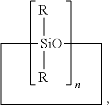

wherein R is independently selected from hydrogen or C₁₋₃₀ straight or branched chain, saturated or unsaturated alkyl, phenyl or aryl, trialkylsiloxy; and where n=3-8 and mixtures thereof. Commonly, a mixture of cyclomethicones is used where n is 4, 5, and/or 6. Commercially available cyclomethicones include Dow Corning UP-1001 Ultra Pure Fluid (i.e. n=4), Dow Corning XIAMETER® PMX-0245 (i.e. n=5), Dow Corning XIAMETER® PMX-0245 (i.e. n=6), Dow Corning 245 fluid (i.e. n=4 and 5), and Dow Corning 345 fluid (i.e. n=4, 5, and 6).

Other silicone oils suitable for use in the personal care composition include polymers having the general formula:

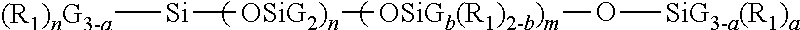

wherein G is hydrogen, phenyl, hydroxy, or C₁-C₈ alkyl, preferably methyl; a is a number 0-3; b is 0 or 1, preferably 1; n is a number from 0 to 1,999 (alternately, from 49 to 499); m is an integer from 1 to 2,000 (alternately, from 1 to 10); the sum of n and m is a number from 1 to 2,000 (alternately, from 50 to 500); R₁ is a monovalent radical conforming to the general formula (CH₂)_{q}L, wherein q is an integer having a value from 1 to 8 and L is selected from the following groups:

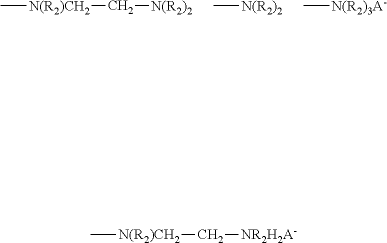

wherein R₂ is hydrogen, phenyl or aryl, or a saturated hydrocarbon radical, preferably an alkyl radical from about C₁ to about C₂₀, and A is a halide ion. An exemplary silicone polymer is trimethylsilylamodimethicone as shown in the following formula:

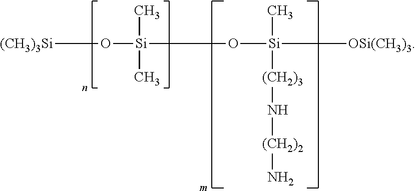

Another exemplary silicone polymer is represented by the general formula:

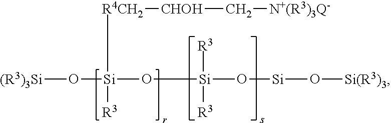

wherein R³ is a monovalent hydrocarbon radical from C₁ to C₁₈, preferably an alkyl or alkenyl, such as methyl; R₄ is a hydrocarbon, preferably a C₁ to C₁₈ alkylene or a C₁₀ to C₁₈ alkyleneoxy, more preferably a C₁ to C₈ alkyleneoxy; Q⁻ is a halide ion, preferably chloride; r is an average statistical value from 2 to 20, preferably from 2 to 8; s is an average statistical value from 20 to 200, preferably from 20 to 50. A suitable polymer of this class is known as UCARE SILICONE ALE 56™, available from Union Carbide. Other suitable silicone materials are disclosed in US Patent Application Publication No. 2007/0039103 A1.

Suitable hydrocarbon oils include straight or branched chain alkanes and alkenes. The chain length may be selected based on desired functional characteristics such as volatility. Suitable hydrocarbon oils may have between 5-20 carbon atoms or, alternately, between 8-16 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105. Suitable hydrocarbons include isooctane, isododecane, isohexadecane, isoeicosane by Permethyl Corporation under the tradename Permethyl®. Suitable hydrocarbon oils may have greater than about 20 carbon atoms. Examples of such hydrocarbon oils include C₂₄₋₂₈ olefins, C₃₀₋₄₅ olefins, C₂₀₋₄₀ isoparaffins, hydrogenated polyisobutene, polyisobutene, polydecene, hydrogenated polydecene, mineral oil, pentahydrosqualene, squalene, squalane, and mixtures thereof.

Other suitable oils include esters. Suitable esters typically contain at least 10 carbon atoms. These esters include esters with hydrocarbyl chains derived from fatty acids or alcohols (e.g., mono-esters, polyhydric alcohol esters, and di- and tri-carboxylic acid esters).

The hydrocarbyl radicals of the esters hereof may include or have covalently bonded thereto other compatible functionalities, such as amides and alkoxy moieties (e.g., ethoxy or ether linkages, etc.). Exemplary esters include, but are not limited to, isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate. Other suitable esters are further described in the Personal Care Product Council's International Cosmetic Ingredient Dictionary and Handbook, Thirteenth Edition, 2010, under the functional category of “Esters.”

Other esters suitable for use in the personal care composition include mono-carboxylic acid esters of the general formula R′COOR, wherein R′ and R are straight or branched chain, saturated or unsaturated alkyl, aryl, and wherein sum of carbon atoms in R′ and R is at least 10, A suitable monoester is alkyl benzoate such as C12-15 alkyl benzoate.

Other esters suitable for use in the personal care composition include di- and tri-alkyl and alkenyl esters of carboxylic acids, such as esters of C₄ to C₈ dicarboxylic acids (e.g. C₁ to C₂₂ esters, preferably C₁ to C₆, of succinic acid, glutaric acid, and adipic acid). Specific non-limiting examples of di- and tri-alkyl and alkenyl esters of carboxylic acids include isocetyl stearyol stearate, diisopropyl adipate, dibutyl adipate, and tristearyl citrate.

Other esters suitable for use in the personal care composition include those known as polyhydric alcohol esters. Such polyhydric alcohol esters include alkylene glycol esters, such as ethylene glycol mono and di-fatty acid esters, diethylene glycol mono- and di-fatty acid esters, polyethylene glycol mono- and di-fatty acid esters, propylene glycol mono- and di-fatty acid esters, polypropylene glycol monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxy ethylene sorbitan fatty acid esters.

Still other esters suitable for use in the personal care composition include glycerides, including, but not limited to, mono-, di-, and tri-glycerides. For use in the compositions described herein, the glycerides may be mono-, di-, and tri-esters of glycerol and long chain carboxylic acids, such as C₁₀ to C₂₂ carboxylic acids. A variety of these types of materials can be obtained from vegetable and animal fats and oils, such as castor oil, safflower oil, cottonseed oil, corn oil, olive oil, cod liver oil, almond oil, avocado oil, palm oil, sesame oil, sweet almond oil, apricot kernel oil, camelina sativa oil, rapeseed oil, tamanu seed oil, linseed oil, coconut oil, lanolin oil, soybean oil, and the like. Synthetic oils include, but are not limited to, triolein and tristearin glyceryl dilaurate. Other glyceryl esters of fatty acids include fatty acid mono-, di-, and triglycerides which are natural fats or oils that have been modified such as glyceryl stearate, diglyceryl diiosostearate, polyglyceryl-3 isostearate, polyglyceryl-4 isostearate, polyglyceryl-6 ricinoleate, glyceryl dioleate, glyceryl diisotearate, glyceryl tetraisostearate, glyceryl trioctanoate, diglyceryl distearate, glyceryl linoleate, glyceryl myristate, glyceryl isostearate. PEG castor oils, PEG glyceryl oleates, PEG glyceryl stearates, PEG glyceryl tallowates, and the like.

Other suitable oils include fatty amides. Fatty amides include compounds having an amide functional group while being liquid at 25° C. and insoluble in water. In certain embodiments, the fatty amide may have the general formula:

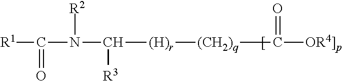

wherein R1 is an optionally functionalized, aliphatic, cycloaliphatic or cyclic, saturated or unsaturated, monovalent hydrocarbon radical containing from 1 to 30 carbon atoms (alternately, from 1 to 22 carbon atoms); R2, R3 and R4, which may be identical or different, are hydrogen or optionally functionalized, aliphatic, cycloaliphatic or cyclic, saturated or unsaturated, monovalent hydrocarbon radicals containing from 1 to 30 carbon atoms, preferably from 1 to 22 carbon atoms; r is 0 or 1: q is an integer from 0 to 2; and p equals 0 or 1. Particular fatty amides include N-acetyl-N-butylaminopropionate, isopropyl N-lauroylsarcosinate, and N,N,-diethyltoluamide. Other suitable fatty amides are disclosed in U.S. Pat. No. 6,872,401.

Other suitable oils include ethers. Suitable ethers include saturated and unsaturated fatty ethers of a polyhydric alcohol, and alkoxylated derivatives thereof. Exemplary ethers include C4-20 alkyl ethers of polypropylene glycols, and di-C8.30 alkyl ethers. Suitable examples of these materials include PPG-14 butyl ether, PPG-15 stearyl ether, dioctyl ether, dodecyl octyl ether, and mixtures thereof.

### Emulsifiers

The personal care composition may comprise an emulsifier. An emulsifier is particularly suitable when the composition is in the form of an emulsion or if immiscible materials are being combined. The personal care composition may comprise from about 0.05%, 0.1%, 0.2%, 0.3%, 0.5%, or 1% to about 20%, 10%, 5%, 3%, 2%, or 1% emulsifier. Emulsifiers may be nonionic, anionic or cationic. Non-limiting examples of emulsifiers are disclosed in U.S. Pat. Nos. 3,755,560, 4,421,769, and McCutcheon's, Emulsifiers and Detergents, 2010 Annual Ed., published by M. C. Publishing Co. Other suitable emulsifiers are further described in the Personal Care Product Council's International Cosmetic Ingredient Dictionary and Handbook, Thirteenth Edition, 2006, under the functional category of “Surfactants—Emulsifying Agents.”

Suitable emulsifying ethers and esters include:

Ethers of polyglycols and of fatty alcohols—including saturated or unsaturated C₁₂₋₃₀ alcohols (e.g., oleyl alcohol, cetyl alcohol, stearyl alcohol or behenyl alcohol) and polyglycols comprising n number of oxyalkylene groups wherein n=an integer from 1 to 200 or, alternately, from 2 to 30 (e.g., 1 to 20 oxyethylene groups). Particular examples include compounds with the INCI names of steareth-n, beheneth-n or oleth-n. Suitable examples include compounds having the INCI names steareth-8, steareth-10, steareth-16, steareth-20, ceteth-10, laureth-4, laureth-3, trideceth-6, ceteareth-5, oleth-10, and beneth-10.

Esters of polyglycols and of fatty acids—including saturated or unsaturated C₁₋₃₀ fatty acids (e.g., oleic acid, cetylic acid, stearic acid) and polyglycols comprising n number of oxyalkylene groups wherein n=an integer from 1 to 200 or alternately, 1 to 50 (e.g., 1 to 20 oxyethylene groups). Particular examples include compounds with the INCI name PEG-n stearate or PEG-n oleate). Suitable examples include polyethylene glycol-8 monostearate, polyethylene glycol-10, or polyethylene glycol-12 distearate.

Ethers of polyglycols and of fatty alcohols which are glycosylated—including C12-30 alcohols having from 1 to 10 glycosyl groups and polyglycols comprising a number of oxyalkylene groups wherein n=an integer from 1 to 200 (e.g., 1 to 20 oxyethylene groups). A suitable example includes polyoxyethylenated (20 OE) methyl glucose distearate.

Esters of polyglycols and of fatty acids which are glycosylated—including C₁₂₋₃₀ fatty acids having from 1 to 10 glycosyl groups and polyglycols comprising a number of oxyalkylene groups wherein n=an integer from 1 to 200 (e.g., 1 to 20 oxyethylene groups).

Ethers of C₁₂₋₃₀ alcohols and of glycerol or of polyglycerol—A suitable example includes polyglyceryl-3 cetyl ether, such as Chimexane NL from Chimex, Esters of C12-30 fatty acids and of glycerol or of polyglycerol—including esters comprising from 1 to 10 glycerol groups. Particular examples include hexa-glyceryl monosterate, diglyceryl distearate, tetraglyceryl tristearate, decaglyceryl decastearate, diglyceryl monostearate, hexaglyceryl tristearate, decaglyceryl pentastearate, the ester of glycerol and of palmitic and stearic acids, and glyceryl mono- and dibehenate.

Ethers of oxyalkylene-modified C12-30 alcohols and of glycerol or polyglycerol.

Ethers of C₁₂₋₃₀ fatty alcohols comprising and of sucrose or glucose—Suitable examples include compounds with the INCI names of C12-18 alkylglucoside, C12-20 alkylglucoside (e.g., Montanov L from Seppic), cetearyl glucoside (e.g., a mixture with cetearyl alcohol under the reference Montanov 68 from Seppic), myristyl glucoside (e.g., a mixture with myristyl alcohol under the reference Montanov 14 from Seppic) or cetearyl glucoside (e.g., Tegocare CG 90 from Evonik Goldschmidt),

Esters of sucrose and of C₁₂₋₃₀ fatty acids—Particular examples include sucrose distearate or sucrose tristearate, sucrose cocoate, sucrose dilaurate, sucrose distearate, sucrose hexaerucate, sucrose hexapalmitate, sucrose laurate, sucrose mortierellate, sucrose myristate, sucrose oleate, sucrose palmitate, sucrose pentaerucate, sucrose polybehenate, sucrose polycottonseedate, sucrose polylaurate, sucrose polylinoleate, sucrose polyoleate, sucrose polypalmate, sucrose polysoyate, sucrose polystearate, sucrose ricinoleate, sucrose stearate, sucrose tetraisostearate, and sucrose trilaurate. A suitable example includes the mixture of esters (mono- and polyesters) of stearic acid and of sucrose sold as Crodesta FI 10 by Croda.

Esters of pentaerythritol and of C₁₂₋₃₀ fatty acids—Particular examples include pentaerythritol tetrastearate.

Esters of sorbitol and/or of sorbitan and of C₁₂₋₃₀ fatty acids—Particular examples include sorbitan monostearate, sorbitan tristearate, or sorbitan laurate, such as Span 20 from Uniqema,

Ethers of sorbitol and/or of sorbitan and of alkoxylated sorbitan-Suitable examples include sorbeth-8 beeswax or sorbeth-20 beeswax from Nikko Chemical.

Ethers of polyglycols and of cholesterol—Particular examples include choleth-3, choleth-10 (such as Emalex CS-10 from Nihon Emulsion Company), choleth-15 (such as Emalex CS-15 from Nihon Emulsion Company) or choleth-20 (such as Emalex CS-20 from Nihon Emulsion Company).

Esters of C₁₂₋₃₀ fatty acids and of alkoxylated ethers of sorbitol and/or of Suitable examples include polysorbate-60, polysorbate-61, sorbeth-3 isostearate, polyoxyethylenated 4 OE sorbitan monostearate, and polyoxyethylenated 20 OE sorbitan tristearate.

Linear or branched type silicone emulsifiers may also be used. Particularly useful polyether modified silicones include KF-6011, KF-6012, KF-6013, KF-6015, KF-6015, KF-6017, KF-6043, KF-6028, and KF-6038 from Shin Etsu. Also particularly useful are the polyglycerolated linear or branched siloxane emulsifiers including KF-6100, KF-6104, and KF-6105 from Shin Etsu. Exemplary materials include materials with the following International Nomenclature of Cosmetic Ingredients (INCI) designations: Bis-Butyldimethicone Polyglyceryl-3; Bis-PEG/PPG-14/14 Dimethicone; Bis-butyldimethicone Polyglyceryl-3; Bis-isobutyl PEG/PPG-10/7 Dimethicone copolymer; Bis-PEG/PPG-18/6 Dimethicone; Bis-PEG/PPG-20/20 Dimethicone; Bis-PEG/PPG-16/16 PEG/PPG-16/16 Dimethicone; Bis(PPG-7 Undeceneth-21-Dimethicone; Cetyl Dimethicone PEG-7 Acetate; Cetyl PEG-8 Dimethicone; Cetyl PEG/PPG-15/16 Butyl Ether Dimethicone; Cetyl PEG/PPG-15/15 Butyl Ether Dimethicone; Cetyl PEG/PPG-7/3 Dimethicone; Cetyl PEG/PPG-10/1 Dimethicone; Dimethicone PEG-15 Acetate; Dimethicone PEG-7 Cocoate; Dimethicone PEG-7 Phosphate; Dimethicone PEG-IO Phosphate; Dimethicone PEG/PPG-7/4 Phosphate: Dimethicone PEG/PPG-12/4 Phosphate; Dimethicone PEG-7 Undecylenate; Lauryl Dimethicone PEG-IO Phosphate; Isopolyglyceryl-3 Dimethicone; Isopolyglyceryl-3 Dimethiconol; Isostearyl Carboxyldecyl PEG-8 Dimethicone; Lauryl Methicone PEG-10 Phosphate; Lauryl PEG-8 Dimethicone; Lauryl PEG-10 Methyl Ether Dimethicone; Lauryl PEG/PPG-18/18 Methicone; PEG-6 Methyl Ether Dimethicone; PEG-7 Methyl Ether Dimethicone; PEG-9 Methyl Ether Dimethicone; PEG-10 Methyl Ether Dimethicone; PEG-11 Methyl Ether Dimethicone: PEG-11 Methyl Ether Dimethicone: PEG-32 Methyl Ether Dimethicone; PEG-PEG/PPG-28/21 Acetate Dimethicone; PEG/PPG-22/22 Butyl Ether Dimethicone; PEG/PPG-23/23 Butyl Ether Dimethicone; PEG/PPG-24/18 Butyl Ether Dimethicone; PEG/PPG-3/10 Dimethicone; PEG/PPG-4/12 Dimethicone; PEG/PPG-6/11 Dimethicone; PEG/PPG-8/14 Dimethicone; PEG/PPG-12/16 Dimethicone; PEG/PPG-12/18 Dimethicone; PEG/PPG-14/4 Dimethicone; PEG/PPG-15/5 Dimethicone; PEG/PPG-15/15 Dimethicone; PEG/PPG-16/2 Dimethicone; PEG/PPG-16/8 Dimethicone; PEG/PPG-17/18 Dimethicone; PEG/PPG-18/12 Dimethicone; PEG/PPG-19/19 Dimethicone; PEG/PPG-20/6 Dimethicone; PEG/PPG-20/15 Dimethicone; PEG/PPG-20/20 Dimethicone; PEG/PPG-20/29 Dimethicone; PEG/PPG-22/23 Dimethicone; PEC/PPG-22/24 Dimethicone; PEG/PPG-25/25 Dimethicone; PEG/PPG-27/27 Dimethicone; PEG/PPG-30/10 Dimethicone; PEG/PPG-10/3 Oleyl Ether Dimethicone; PEG-8 trisiloxane; Polyglyceryl-3 Polydimethylsiloxyethyl Dimethicone; PPG-12 Butyl Ether Dimethicone; Silicone Quaternium-17; TEA-Dimethicone PEG-7 Phosphate; and mixtures thereof.

Emulsifiers also include emulsifying silicone elastomers. Suitable emulsifying silicone elastomers may include at least one polyalkyl ether or polyglycerolated unit. These cross-linked elastomers may also be co-modified to include alkyl substituents. Suitable formation techniques are described in U.S. Pat. Nos. 5,236,986; 5,412,004; 5,837,793; and 5,811,487. Polyoxyalylenated emulsifying silicone elastomers that may be used in at least one embodiment of the disclosure include those sold by Shin-Etsu Silicones under the names KSG-21, KSG-20, KSG-30, KSG-31, KSG-32, KSG-33; KSG-210 (dimethicone/PEG-10/15 crosspolymer dispersed in dimethicone); KSG-310 (PEG-15 lauryl dimethicone crosspolymer); KSG-320 (PEG-15 lauryl dimethicone crosspolymer dispersed in isododecane); KSG-330 (PEG-15 lauryl dimethicone crosspolymer dispersed in triethylhexanoin), KSG-340 (PEG-10 lauryl dimethicone crosspolymer and PEG-15 lauryl dimethicone crosspolymer). Other silicone emulsifying elastomers are supplied by Dow Corning™, including PEG-12 dimethicone crosspolymers (DC 9010 and 9011). Other suitable silicone emulsifiers sold by Dow Corning include DC9010 and DC9011.

Polyglycerolated emulsifying silicone elastomers are disclosed in PCT/WO 2004/024798. Such elastomers include Shin-Etsu's KSG series, such as KSG-710 (dimethicone/polyglycerin-3 crosspolymer dispersed in dimethicone); or lauryl dimethicone/polyglycerin-3 crosspolymer dispersed in a variety of solvent such as isododecane, dimethicone, triethylhexanoin, available as KSG-810, KSG-820, KSG-830, or KSG-840 from Shin-Etsu.

Another suitable crosslinked silicone elastomer emulsifier is dimethicone/PEG-10/15 crosspolymer, which provides excellent aesthetics due to its elastomeric backbone, but also excellent emulsification properties. Further examples of crosslinked organosiloxane emulsifiers include, but are not limited to dimethicone/dimethicone PEG/PPG 15 crosspolymer; dimethicone PEG-10 crosspolymer; dimethicone PEG-10/15 crosspolymer; dimethicone PEG-15 crosspolymer; dimethicone polyglycerin-3 crosspolymer; dimethicone PPG-20 crosspolymer; lauryl dimethicone PEG-15 crosspolymer; lauryl dimethicone polyglycerin-3 crosspolymer; PEG-8 dimethicone polysorbate-20 crosspolymer; PEG-10 dimethicone/vinyl dimethicone crosspolymer; PEG-10 lauryl dimethicone crosspolymer; PEG-15/lauryl dimethicone crosspolymer; PEG-15 laurylpolydimethylsiloxy ethyl crosspolymer; and mixtures thereof.

It should be recognized that silicone elastomers may be supplied pre-swollen with a solvent. With a pre-swollen swollen elastomer, the weight percentages recited for emulsifier use (i.e., from about 0.05% to about 20%, from about 0.1% to about 10%, from about 0.5% to about 5%, or from about 1% to about 3% emulsifier) are of the elastomer alone (i.e., excluding the weight of the solvent).

### Structuring Agent

The personal care composition may comprise a structuring agent. Structuring agents may be used to increase viscosity, thicken, solidify, or provide solid or crystalline structure to the personal care composition. The structuring agent may be used to suspend or disperse the abrasive particles. Structuring agents are typically grouped based on solubility, dispersibility, or phase compatibility. Examples of aqueous or water structuring agents include polymeric agents, natural or synthetic gums, polysaccharides, and the like. In one embodiment, the composition may comprises from about 0.0001%, 0.001%, 0.01%, 0.05%, 0.1%, 0.5%, 1%, 2%, 3%, 5% to about 25%, 20%, 10%, 7%, 5%, 4%, or 2%, by weight of the composition, of one or more structuring agents.

Polysaccharides and gums may be used as aqueous phase thickening agents. Examples of such polysaccharides and gums include naturally derived materials such as agar, agarose, alicaligenes polysaccharides, algin, alginic acid, acacia gum, amylopectin, chitin, dextran, cassia gum, cellulose gum, gelatin, gellan gum, hyaluronic acid, hydroxyethyl cellulose, methyl cellulose, ethyl cellulose, pectin, sclerotium gum, xanthan gum, pectin, trehelose, gelatin, ammonium alginate, calcium alginate, calcium carrageenan, carnitine, carrageenan, guar gum, guar hydroxypropyltrimonium chloride, hyaluronic acid, hydroxypropyl chitosan, hydroxypropyl guar, karaya gum, kelp, locust bean gum, natto gum, potassium alginate, potassium carrageenan, propylene glycol alginate, sodium carboyxmethyl dextran, sodium carrageenan, tragacanth gum, and mixtures thereof. Suitable polysaccharides include alkyl hydroxyalkyl cellulose ethers such as cetyl hydroxyethylcellulose, which is the ether of cetyl alcohol and hydroxyethylcellulose. This material is sold under the tradename Natrosol® Plus CS from Ashland Aqualon Functional Ingredients. Other useful polysaccharides include scleroglucans comprising a linear chain of (1-3) linked glucose units with a (1-6) linked glucose every three units, a commercially available example of which is Clearogel™ CS 11 from M.M.P., Inc.

Suitable classes of polymeric structuring agents include but are not limited to carboxylic acid polymers, polyacrylamide polymers, sulfonated polymers, high molecular weight polyalkylglycols or polyglycerins, copolymers thereof, hydrophobically modified derivatives thereof, and mixtures thereof.

Carboxylic acid polymers include carbomers. These polymers are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol. Suitable materials includes include the Carbopol® 900 series (e.g., Carbopol® 945, Carbopol® 940, Carbopol® 950, Carbopol® 954, Carbopol®) 980, Carbopol® 951 and Carbopol® 981 from Noveon, Inc) and the Carbopol® Ultrez series (e.g., Carbopol® Ultrez 10 polymer, Carbopol® Ultrez 20 polymer, and Carbopol® Ultrez 21 polymer). Other suitable carboxylic acid polymeric agents include copolymers of Co10-30 alkyl acrylates with one or more monomers of acrylic acid, methacrylic acid, or one of their short chain (i.e., C₁-4 alcohol) esters, wherein the crosslinking agent is an allyl ether of sucrose or pentaerytritol. These copolymers are known as acrylates/C₁₀₋₃₀ alkyl acrylate crosspolymers and are commercially available as Carbopol® 1342, Carbopol® 1382, PEMULEN™ TR-1, and PEMULEN™ TR-2, from Noveon, Inc.

Sulfonated polymers include polymers and copolymers containing 2-acrylamido-2-methylpropane sulfonic acid (i.e., AMPS or acryloyldimethyl tauric acid) and salts thereof. Exemplary AMPS structurants include sodium acrylate/sodium acryloyldimethyl taurate copolymer available as SIMULGEL® EG and SIMULGEL® EPG or hydroxyethyl acrylate/sodium acryloyldimethyl taurate copolymer available as SIMULGEL® NS, SIMULGEL® FL, and SIMULGEL® I-NS 100; which are available from Seppic Corporation (Fairfield, N.J.). Another suitable sulfonated polymer is sodium polyacryloyldimethyl taurate available as Simulgel® 800 from Seppic Corporation (Fairfield, N.J.), Other suitable sulfonated polymers include acrylamide/sodium acryloyldimethyltaurate/acrylic acid copolymer available as Acudyne™ SCP from Rohm and Haas Company, Inc.; acrylamide/sodium acryloyldimethyltaurate copolymer available as Simulgel® 600 from Seppic; ammonium acryloyldimethyltaurate/beheneth-25 methacrylate crosspolymer avilable as Aristoflex® BLV from Clariant International Ltd.; ammonium acryloyl dimethyltaurate/carboxyethyl acrylate crosspolymer avilable as Aristoflex® TAC from Clariant International Ltd.; ammonium acryloyldimethyltaurate/vinylpyrrolidone copolymer available as Aristoflex® AVC from Clariant International Ltd.; dimethylacrylamide/sodium acryloyldimethyltaurate crosspolymer available as SUpolymer G-1 from Toho Chemical Industry Co., Ltd.; sodium acrylate/acryloyldimethyltaurate/dimethylacrylamide crosspolymer available as Sepinov™ P88 from Seppic; and sodium acryloyldimethyltaurate/VP Crosspolymer available as Aristoflex® AVS from Clariant International, Ltd. Additional sulfonated structurants are described in US Patent Application Publication Nos. 2007/0140993 (identified as gelling agent in the form of a copolymer of acryloyl dimethyl tauric acid or a salt thereof) and 2006/0147396 A1 (identified as “polymer containing at least one sulpho-functional monomer”).

Acrylamide polymers and copolymers include SEPIGEL® 305 from Seppic Corporation (Fairfield, N.J.), which is designated by the Personal Care Product Council's International Cosmetic Ingredient Dictionary and Handbook, Thirteenth Edition, 2010, as “polyacrylamide and isoparaffin and laureth-7.” Other polyacrylamide polymers include multi-block copolymers of acrylamides and substituted acrylamides with acrylic acids and substituted acrylic acids. Commercially available examples of these multi-block copolymers include HYPAN® SR150H, SS500V, SS500 W, SSSA100H, from Lipo Chemicals, Inc., (Patterson, N.J.).

High molecular weight polyalkylglycols or polyglycerins may be used as structuring agents. Suitable materials include polyethylene glycols (PEC) derivatives and polypropylene glycols (PPG) derivatives with an n degree of polymerization. n may be from 50 to 200,000. Other suitable materials are polyglycerins having repeating glycerin moieties where the number of repeating moieties ranges from about 15 to about 200, or from about 20 to about 100. Examples of suitable polyglycerins include those having the INCI names polyglycerin-20, polyglycerin-40, and the like.

Examples of oil structuring agents include silicone and organic based materials. Suitable ranges of oil structuring agents are from about 0.01%, 0.05%, 0.1% 0.5%, 1%, 2.5%, 5%, or 10% to about 30%, 25%, 20%, 15%, 10%, or 5%. Suitable oil phase structuring agents may be silicone based, such as silicone elastomers, silicone gums, silicone waxes, linear silicones having a degree of polymerization allowing the silicone to increase the viscosity of the oil phase. Examples of silicone structuring agents include, but are not limited to, silicone elastomers, silicone gums, and silicone waxes.

Silicone elastomers suitable for use in the compositions of the disclosure include those that are formed by addition reaction-curing, by reacting an SiH-containing diorganosiloxane and an organopolysiloxane having terminal olefinic unsaturation, or an alpha-omega diene hydrocarbon, in the presence of a platinum metal catalyst. Such elastomers may also be formed by other reaction methods such as condensation-curing organopolysiloxane compositions in the presence of an organotin compound via a dehydrogenation reaction between hydroxyl-terminated diorganopolysiloxane and SiH-containing diorganopolysiloxane or alpha omega diene; or by condensation-curing organopolysiloxane compositions in the presence of an organotin compound or a titanate ester using a condensation reaction between an hydroxyl-terminated diorganopolysiloxane and a hydrolysable organosiloxane; peroxide-curing organopolysiloxane compositions which thermally cure in the presence of an organoperoxide catalyst. Cross-linked organopolysiloxane elastomers useful in the present disclosure and processes for making them are further described in U.S. Pat. Nos. 4,970,252, 5,760,116, and 5,654,362, 6,524,598, and 6,696,049. It is particularly desirable to incorporate silicone elastomers into the compositions of the disclosure because they provide excellent “feel” to the composition, are very stable in cosmetic formulations, and relatively inexpensive.

Suitable silicone elastomers may be in the powder form, or dispersed or solubilized in solvents such as volatile or nonvolatile silicones, or silicone compatible vehicles such as hydrocarbons or esters. Examples of silicone elastomer powders include vinyl dimethicone/methicone silesquioxane crosspolymers like KSP-100, KSP-101, KSP-102, KSP-103, KSP-104, KSP-105, available from Shin-Etsu, hybrid silicone powders that contain a fluoroalkyl group like KSP-200, available from Shin-Etsu, which is a fluoro-silicone elastomer, and hybrid silicone powders that contain a phenyl group such as KSP-300, available from Shin-Etsu, which is a phenyl substituted silicone elastomer; and DC 9506 available from Dow Corning.

Examples of silicone elastomer dispersed in a silicone compatible vehicle include dimethicone/vinyl dimethicone crosspolymers supplied by a variety of suppliers including Dow Corning Corporation under the tradenames DC9040 or DC9041, Momentive under the tradename SFE 839, or Shin-Etsu Silicones under the tradenames KSG-15, 16, 18. KSG-15 has the INCI name cyclopentasiloxane (and) dimethicone/vinyl dimethicone crosspolymer. KSG-18 has the INCI name diphenylsiloxy phenyl trimethicone (and) dimethicone/phenyl vinyl dimethicone crosspolymer. Silicone elastomers may also be purchased from Grant Industries under the Gransil trademark. Other suitable silicone elastomers have long chain alkyl substitutions such as lauryl dimethicone/vinyl dimethicone crosspolymers supplied by Shin Etsu under the tradenames KSG-31, KSG-32, KSG-41, KSG-42, KSG-43, and KSG-44.

Silicone gums are another oil phase structuring agent. The silicone gum typically has a viscosity ranging from about 500,000 to 100 million cst at 25° C., from about 600,000 to 20 million, from about 600,000 to 12 million est. The silicone gums that are used in the compositions include, but are not limited to, those of the general formula wherein:

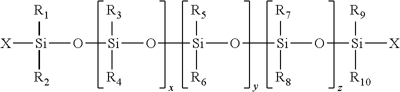

R₁ to R₁₀ are each independently hydrogen, an alkyl having 1 to 30 carbon atoms, aryl, or aralkyl; and X is H, OH, or a C1-30 alkyl or vinyl. x, y, or z may be zero with the proviso that (x+y+z)≥1.

Such silicone gums may be purchased in pure form from a variety of silicone manufacturers including Wacker-Chemie or Dow Corning, and the like. Silicone gums include those sold by Wacker-Belsil under the trade names CM3092, Wacker-Belsil 1000, or Wacker Belsil DM 3096. A silicone gum where X is OH, also referred to as dimethiconol, is available from Dow Corning Corporation under the trade name 1-1254 Fluid, 2-9023 Fluid, and 2-9026 Fluid. The silicone gum may also be purchased in the form of a solution of dispersion in a silicone compatible vehicle such as volatile or nonvolatile silicone. An example of such a mixture may be purchased from Bamet Silicones under the HL-88 tradename, having the INCI name dimethicone. Another example is a mixture of dimethiconol and volatile or nonvolatile silicone available from the Dow Corning Corporation as tradename 1401 Fluid, 1403 Fluid, and 1501 Fluid.

Another type of oily phase structuring agent includes silicone waxes. Silicone waxes may be referred to as alkyl silicone waxes which and are semi-solids or solids at room temperature. The term “alkyl silicone wax” means a polydimethylsiloxane having a substituted long chain alkyl (such as C16 to 30) that confers a semi-solid or solid property to the siloxane. Examples of such silicone waxes include stearyl dimethicone, which may be purchased from Evonik Goldschmidt GmbH under the tradename Abil Wax 9800 or from Dow Corning under the tradename 2503. Another example is bis-stearyl dimethicone (which may be purchased from Gransil Industries under the tradename Gransil A-18), behenyl dimethicone, or behenoxy dimethicone.

Other suitable structuring agents include polyamides and polysilicone-polyamide copolymers. Suitable polysilicone-polyamide copolymers are disclosed in U.S. Patent Application Publication No. 2004/0170586. A specific example of such copolymers is nylon 611/dimethicone copolymers by Dow Corning under the tradename Dow Corning 2-8178. Also suitable are polyamides such as those purchased from Arizona Chemical under the Uniclear™ and Sylvaclear® including Sylvaclear® A200V or A2614V (INCI name: ethylenediamine/hydrogenated dimer dilinoleate copolymer/bis-di-C14-18 alkyl amide): Sylvaclear® AF1900V and Sylvaclear® PA1200V (INCI name: Polyamide-3); Sylvaclear® C75V (INCI name: bis-stearyl ethylenediamine/neopentyl glycol/stearyl hydrogenated dimer dilinoleate copolymer); Sylvaclear® PE400V (INCI name: Polyamide-6); Sylvaclear®) WF 1500V (INCI name: Polyamide-4); or Uniclear™ 100 VG (INCI name: ethylenediamine/stearyl dimer dilinoleate copolymer; or ethylenediamine/stearyl dimer ditallate copolymer).

Other oil phase structuring agents may include one or more natural or synthetic waxes such as animal, vegetable, or mineral waxes. Generally such waxes have a melting point ranging from about 25° C. to 125° C., and alternatively from about 30° C. to about 100° C. Non-limiting examples of suitable waxes include silicone waxes, fatty esters, for example cetyl and/or stearyl esters, acacia, beeswax, ceresin, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, candelilla, grape wax, polyalkylene glycol derivatives thereof (for example PEG6-20 beeswax, or PEG-12 carnauba wax) and mixtures of any of the aforementioned waxes. In one embodiment, the wax is a polyethylene wax, and alternatively is a polyethylene wax having a melting point of less than 120° C., alternatively less than 95 C, and alternatively less than 85° C.

Non-limiting examples of suitable silicone waxes are disclosed in U.S. Pat. Nos. 5,413,781 and 5,725,845, and further include alkylmethyl polysiloxanes, C10-C60 alkyl dimethicones, and mixtures thereof. Alternatively, the silicone wax may be a C16-C28 alkyl dimethicone wax. Other suitable silicone waxes include, but are not limited to stearoxydimethicone, behenoxy dimethicone, stearyl dimethicone, cetearyl dimethicone, cetyl dimethicone, and mixtures thereof.

Other structuring agents are natural or synthetic montmorillonite minerals such as hectorite, bentonite, and quaternized derivatives thereof, which are obtained by reacting the minerals with a quaternary ammonium compound (e.g., stearalkonium bentonite and stearalkonium hectorite).

Other structuring agents are silicas, silicates, silica silylate, and alkali metal or alkaline earth metal derivatives thereof. These silicas and silicates are generally found in the particulate form and include silica, silica silylate, magnesium aluminum silicate, and the like.

### Additional Ingredients

The personal care compositions may comprise one or more additional components to provide an efficacious and/or consumer desirable product. For example, the composition can include other actives or agents. For instance, suitable optional actives and agents may include an active or agent selected from a group consisting of sugar amines, vitamins, oil control agents, photosterols, hexamidine compounds, tightening agents, anti-wrinkle actives, anti-atrophy actives, flavonoids, N-acyl amino acid compounds, retinoids, peptides, particulate materials, UV actives, photostabilizers, anti-cellulite agents, desquamation actives, anti-acne actives, anti-oxidants, radical scavengers, conditioning agents, anti-inflammatory agents, tanning actives, skin lightening agents, botanical extracts, antimicrobial actives, antifungal actives, antibacterial actives, antiperspirant actives, sensates, preservatives, anti-dandruff actives, substantivity polymers, detersive surfactants, and combinations thereof. Suitable non-limiting examples are discussed in more detail below.

### 1. Sugar Amines

The personal care compositions of the present disclosure can comprise a sugar amine, which is also known as amino sugar. Sugar amine compounds useful in the present disclosure can include those described in PCT Publication WO 02/076423 and U.S. Pat. No. 6,159,485. In one embodiment, the composition may comprise from about 0.001%, 0.01%, 0.05%, 0.1%. 0.5%, or 1% to about 10%, 7, 5%, or 2% by weight of the composition, of one or more sugar amine.

Sugar amines can be synthetic or natural in origin and can be used as pure compounds or mixtures of compounds (e.g., extracts from natural sources or mixtures of synthetic materials). For example, glucosamine is generally found in many shellfish and can also be derived from fungal sources. As used herein, “sugar amine” includes isomers and tautomers of such and its salts (e.g., HCl salt) and is commercially available from Sigma Chemical Co.

Examples of sugar amines that are useful herein include glucosamine, N-acetyl glucosamine, mannosamine, N-acetyl mannosamine, galactosamine, N-acetyl galactosamine, their isomers (e.g., stereoisomers), and their salts (e.g., HCl salt). Preferred for use herein are glucosamine, particularly D-glucosamine and N-acetyl glucosamine, particularly N-acetyl-D-glucosamine.

### 2. Vitamins

In some embodiments, the composition may comprise from about 0.001%, 0.01%. 0.05%, 0.1%, 0.5%, or 1% to about 10%, 7, 5%, or 2%, by weight of the composition, of one or more vitamins. “Vitamins” means vitamins, pro-vitamins, and their salts, isomers and derivatives. Non-limiting examples of suitable vitamins include: vitamin B compounds (including B1 compounds, B2 compounds, B3 compound, B5 compounds, such as panthenol or “pro-B5”, pantothenic acid, pantothenyl: B6 compounds, such as pyroxidine, pyridoxal, pyridoxamine; carnitine, thiamine, riboflavin); vitamin A compounds, and all natural and/or synthetic analogs of Vitamin A, including retinoids, retinol, retinyl acetate, retinyl palmitate, retinoic acid, retinaldehyde, retinyl propionate, carotenoids (pro-vitamin A), and other compounds which possess the biological activity of Vitamin A; vitamin D compounds; vitamin K compounds; vitamin E compounds, or tocopherol, including tocopherol sorbate, tocopherol acetate, other esters of tocopherol and tocopheryl compounds; vitamin C compounds, including ascorbate, ascorbyl esters of fatty acids, and ascorbic acid derivatives, for example, ascorbyl phosphates such as magnesium ascorbyl phosphate and sodium ascorbyl phosphate, ascorbyl glucoside, and ascorbyl sorbate; and vitamin F compounds, such as saturated and/or unsaturated fatty acids.

In certain embodiments, the personal care compositions comprise a vitamin B3 compound.

Vitamin B3 compounds are particularly useful for regulating skin conditions, as described in U.S. Pat. No. 5,939,082, the disclosure of which is incorporated herein in its entirety by reference. In one embodiment, the composition may comprise from about 0.001%, 0.01%, 0.05%, 0.1%, 0.5%, or 1% to about 50%, 20%, 10%, 7%, or 5%, by weight of the composition, of the vitamin B3 compound.

As used herein, “vitamin B3 compound” means a compound having the formula:

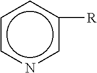

wherein R is —CONH₂ (i.e., niacinamide), —COOH (i.e., nicotinic acid) or —CH2OH (i.e., nicotinyl alcohol); derivatives thereof; and salts of any of the foregoing.

Exemplary derivatives of the foregoing vitamin B3 compounds include nicotinic acid esters, including non-vasodilating esters of nicotinic acid (e.g., tocopherol nicotinate, myristyl nicotinate), nicotinyl amino acids, nicotinyl alcohol esters of carboxylic acids, nicotinic acid N-oxide and niacinamide N-oxide.

In certain embodiments, the personal care compositions comprise a vitamin C compound. In some embodiments, the vitamin C compound is a vitamin C derivative.

In some embodiments, the vitamin C derivative is ascorbyl palmitate having the formula:

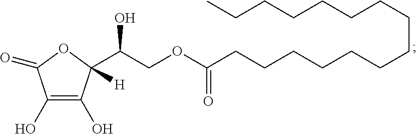 [SMILES: CCCCCCCCCCCCCCCC(=O)OC[C@H](O)[C@H]1OC(=O)C(O)=C1O]

derivatives thereof; and salts of any of the foregoing.

In some embodiments, the vitamin C derivative is magnesuim ascorbyl phosphate having the formula:

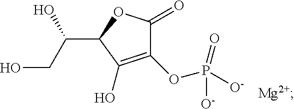 [SMILES: O=C1O[C@H]([C@@H](O)CO)C(O)=C1OP(=O)([O-])[O-]]

derivatives thereof; and salts of any of the foregoing.

In some embodiments, the vitamin C derivative is ethyl ascorbic acid having the formula:

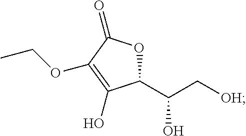

derivatives thereof; and salts of any of the foregoing.

In some embodiments, the vitamin C derivative is ascorbyl glucoside (AA-2G) having the formula:

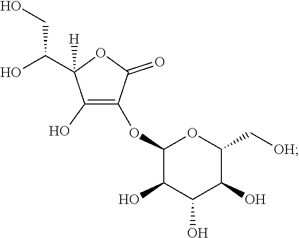 [SMILES: O=C1O[C@H]([C@H](O)CO)C(O)=C1O[C@H]1O[C@H](CO)[C@@H](O)[C@H](O)[C@H]1O]

derivatives thereof; and salts of any of the foregoing.

### 3. Phytosterols

The personal care compositions may comprise a phytosterol. For example, one or more phytosterols can be selected from the group consisting of -sitosterol, campesterol, brassicasterol, A5-avennasterol, lupenol, α-spinasterol, stigmasterol, their derivatives, analogs, and combinations thereof. In certain embodiments, the phytosterol is selected from the group consisting of 3-sitosterol, campesterol, brassicasterol, stigmasterol, their derivatives, and combinations thereof. In a select embodiment, the phytosterol is stigmasterol.

Phytosterols can be synthetic or natural in origin and can be used as essentially pure compounds or mixtures of compounds (e.g., extracts from natural sources). Phytosterols are generally found in the unsaponifiable portion of vegetable oils and fats and are available as free sterols, acetylated derivatives, sterol esters, ethoxylated or glycosidic derivatives. More preferably, the phytosterols are free sterols. As used herein, “phytosterol” includes isomers and tautomers of such and is commercially available from Aldrich Chemical Company, Sigma Chemical Company, and Cognis.

In one embodiment, the composition may comprise from about 0.0001%, 0.001%. 0.01%, 0.05%, 0.1%, 0.5%, or 1% to about 25%, 20%, 10%, 7%, 5%, or 3%, by weight of the composition, of one or more phytosterol.

### 4. Hexamidine Compounds

The personal care compositions may include hexamidine compounds, its salts, and derivatives. As used herein, “hexamidine compound” means a compound having the formula:

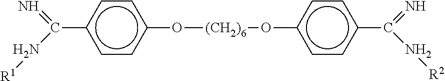

wherein R¹ and R² are optional or are organic acids (e.g., sulfonic acids, etc.).

In one embodiment, the composition may comprise from about 0.0001%, 0.001%, 0.01%, 0.05%, 0.1%, 0.5%, or 1% to about 25%, 20%, 10%, 7%, 5%, or 3%, by weight of the composition, of one or more hexamine compounds.

As used herein, hexamidine derivatives include any isomers and tautomers of hexamidine compounds including but not limited to organic acids and mineral acids, for example sulfonic acid, carboxylic acid, etc. In a select embodiment, the hexamidine compounds include hexamidine diisethionate, commercially available as Eleastab® HP100 from Laboratories Serobiologiques.

### 5. Tightening Agents

The personal care composition may comprise a tightening agent. A tightening agent is a compound capable of having a tightening effect on keratinous tissues and, typically, on skin. Suitable tightening agents may be chosen from plant or animal proteins and their hydrolysates such as maize, rye, wheat, buckwheat, sesame, spelt, pea, bean, lentil, soybean and lupin; polysaccharides of natural origin including (i) polyholosides, for example, in the form of starch derived especially from rice, maize, potato, cassava, peas, wheat, oats, etc. or in the form of carrageenans, alginates, agars, gellans, cellulose polymers and pectins, advantageously as an aqueous dispersion of gel microparticles, and (ii) latices composed of shellac resin, gum sandarac, dammars, elemis, copals, cellulose compounds, and mixtures thereof; mixed silicates including phyllosilicates and in particular laponites; colloidal particles of inorganic fillers such as silica/alumina colloidal particles such as those sold under then tradename LUDOX® by W.R. Grace & Co.; synthetic polymers such as polyurethane latices or acrylic/silicone latices, in particular those described in US Patent Application Publication No. 2002/0131948, including propylthio(polymethyl acrylate), propylthio(polymethyl methacrylate) and propylthio(polymethacrylic acid) grafted polydimethylsiloxane, propyl-thio (polyisobutyl methacrylate) and propylthio(poly-methacrylic acid) grafted polydimethylsiloxane (available under the tradenames VS 80, VS 70 and 1.021 from 3M); and mixtures thereof.

The personal care composition may comprise from about 0.0001%, 0.001%, 0.01%, 0.05%, 0.1%, 0.5%, or 1% to about 30%, 25%, 20%, 10%, 7%, 5%, or 3% by weight of the composition, of one or more tightening agent.

### 6. Anti-Wrinkle Actives/Anti-Atrophy Actives

The personal care compositions of the present disclosure can comprise a one or more anti-wrinkle actives or anti-atrophy actives. Exemplary anti-wrinkle/anti-atrophy actives suitable for use in the compositions of the present disclosure include dialkanoyl hydroxyproline compounds, hydroxy acids (e.g., glycolic acid, lactic acid, lactobionic acid), keto acids (e.g., pyruvic acid), phytic acid, lysophosphatidic acid, stilbenes, cinnamates, resveratrol, kinetin, zeatin, dimethylaminoethanol, peptides from natural sources (e.g., soy peptides), and salts of sugar acids (e.g., Mn gluconate, Zn gluconate). In one embodiment, the composition may comprise from about 0.0001%, 0.001%, 0.01%, 0.05%, 0.1%, 0.5%, or 1% to about 30%, 25%, 20%, 10%, 7%, 5%, or 3% by weight of the composition, of one or more anti-wrinkle/anti-atrophy compounds.

Suitable dialkanoyl hydroxyproline compounds of the present disclosure can include those corresponding to the following chemical formula:

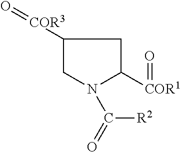

wherein R¹ is H, X, C₁-C₂₀ straight or branched alkyl,
X is metals (Na, K, Li, Mg, Ca) or amines (DEA, TEA);
R² is C₁-C₂₀ straight or branched alkyl;
R³ is C₁-C₂₀ straight or branched alkyl.

Suitable derivatives include but are not limited to esters, for example fatty esters, including, but not limited to tripalmitoyl hydroxyproline and dipalmityl acetyl hydroxyproline. A particularly useful compound is dipalmitoyl hydroxyproline. As used herein, dipalmitoyl hydroxyproline includes any isomers and tautomers of such and is commercially available under the tradename Sepilift DPHP® from Seppic, Inc. Further discussion of dipalmitoyl hydroxyproline appears in PCT Publication WO 93/23028. Preferably, the dipalmitoyl hydroxyproline is the triethanolamine salt of dipalmitoyl hydroxyproline as discussed in U.S. Pat. No. 7,285,570.

### 7. Flavonoids

The personal care compositions of the present disclosure can comprise a flavonoid compound. Flavonoids are broadly disclosed in U.S. Pat. Nos. 5,686,082 and 5,686,367. Examples of flavonoids particularly suitable for use in the present disclosure are one or more flavones, one or more isoflavones, one or more coumarins, one or more chromones, one or more dicoumarols, one or more chromanones, one or more chromanols, isomers (e.g., cis/trans isomers) thereof, and mixtures thereof.

Exemplary flavonoids include flavones and isoflavones, in particular daidzein (7,4′-dihydroxy isoflavone), genistein (5,7,4′-trihydroxy isoflavone), equol (7,4′-dihydroxy isoflavan), 5,7-dihydroxy-4′-methoxy isoflavone, soy isoflavones (a mixture extracted from soy) and other plant sources of such mixtures (e.g., red clover), and mixtures thereof. Other exemplary materials include flavanones such as hesperitin, hesperidin, and mixtures thereof. Flavonoid compounds useful herein are commercially available from a number of sources, e.g., Indofine Chemical Company, Inc., Steraloids, Inc., and Aldrich Chemical Company, Inc.

In one embodiment, the composition may comprise from about 0.0001%, 0.001%, 0.01%, 0.05%, 0.1%, 0.5%, or 1% to about 30%, 25%, 20%, 10%, 7%, 5%, or 3%, by weight of the composition, of one or more flavonoid compounds.

### 8. Particulate Materials

The personal care compositions of the present disclosure can comprise one or more additional particulate materials. Nonlimiting examples of particulate materials useful in the present disclosure include colored and uncolored pigments, interference pigments, inorganic powders, organic powders, composite powders, optical brightener particles, and combinations thereof. In one embodiment, the composition may comprise from about 0.0001%, 0.001%, 0.01%, 0.05%. 0.1%, 0.5%, 1%, or 2% to about 50%, 25%, 20%, 10%, 7%, 5%, or 3% by weight of the composition, of particulate(s). There are no specific limitations as to the pigment, colorant or filler powders used in the composition.

Particulate materials useful herein can include, but are not limited to, bismuth oxychloride, sericite, mica, mica treated with barium sulfate or other materials, zeolite, kaolin, silica, boron nitride, lauroyl lysine, nylon, polyethylene, tale, styrene, polypropylene, polystyrene, ethylene/acrylic acid copolymer, polyurethane, aluminum oxide, silicone resin, barium sulfate, calcium carbonate, cellulose acetate, PTFE, polymethyl methacrylate, starch, modified starches such as aluminum starch octenyl succinate, silk, glass, and mixtures thereof. Suitable commercial examples of particulates include, but are not limited, to polymeric particles chosen from the polymethylsilsesquioxane resin microspheres such as including materials sold under the tradename Tospearl® by Momentive Performance Materials Inc., microspheres of polymethylmethacrylates such Micropearl M305 by SEPPIC, spherical particles of crosslinked polydimethylsiloxanes, especially such as those sold by Dow Corning 9506 Cosmetic Power by Dow Corning, spherical particles of polyamide and more specifically Nylon 12 such as Orgasol® 2002 line by Atochem, polystyrene microspheres such as for example those sold under the name Dynospheres® by Dyno Particles, ethylene acrylate copolymer sold under the name EA209 by Kobo, PTFE, polypropylene, aluminum starch ocetenylsuccinate such as those sold under the name Dry-Flo® by AkzoNobel, microspheres of polyethylene such as those sold under the name of Microthene® FN510-00 by Equistar and under the name Micropoly by Presperse, Inc., silicone resin, polymethylsilsesquioxane silicone polymer, and mixtures thereof. Suitable particulate materials include spherical powders with an average primary particle size of from about 0.1 to about 75 microns or from about 0.2 to about 30 microns.

Other suitable particulate materials include interference pigments. Interference pigments, for purposes of the present specification, are defined as thin platelike layered particles having two or more layers of controlled thickness with different refractive indices that yield a characteristic reflected color from the interference of typically two, but occasionally more, light reflections, from different layers of the platelike particle. The most common examples of interference pigments are micas layered with about 50-300 nm films of TiO₂, Fe₂O₃, silica, tin oxide, and/or Cr₂O₃. Such pigments are often pearlescent. Pearl pigments reflect, refract and transmit light because of the transparency of pigment particles and the large difference in the refractive index of mica platelets and, for example, the titanium dioxide coating. Useful interference pigments are available commercially from a wide variety of suppliers, for example, Rona (Timiron™ and Dichrona™), Presperse (Flonac™), Englehard (Duochrome™), Kobo (KTZ Interfine and KTZ Interval), BASF (Reflecks™) and Eckart (Prestige series). Suitable interference pigments may have a small particle sizes, with an average diameter of individual particles less than about 75 microns in the longest direction, or less than about 50 microns.

Other particulate materials include pigments which can provide color to the personal care composition. Suitable pigments include inorganic pigments, organic pigments and combinations thereof. Examples of such useful inorganic pigments include iron oxides, ferric ammonium ferrocyanide, manganese violet, ultramarine blue, and Chrome oxide. Organic pigments can include natural colorants and synthetic monomeric and polymeric colorants. An example is phthalocyanine blue and green pigment. Also useful are lakes, primary FD&C or D&C lakes and blends thereof. Also useful are encapsulated soluble or insoluble dyes and other colorants. Inorganic white or uncolored pigments useful in the present disclosure, for example TiO₂, ZnO, or ZrO₂, are commercially available from a number of sources. One example of a suitable particulate material contains the material available from U.S. Cosmetics (TRONOX TiO2 series, SAT-T CR837, a rutile TiO2). Suitable pigments include charged dispersions of titanium dioxide, as are disclosed in U.S. Pat. No. 5,997,887.

Colored or uncolored pigments may have a primary average particle size of from about 10 nm, 15 nm, or 20 nm to about 100,000 nm, 5,000 nm, or 1000 nm. Mixtures of the same or different pigments having different particle sizes are also useful herein (e.g., incorporating a TiO₂ having a primary particle size of from about 100 nm to about 400 nm with a TiO₂ having a primary particle size of from about 10 nm to about 50 nm).

The particulate materials can be surface treated to provide added stability and/or for ease of formulation. Non-limiting examples of suitable coating materials include silicones, lecithin, amino acids, metal soaps, polyethylene and collagen. These surface treatments may be hydrophobic or hydrophilic. Particularly useful hydrophobic pigment treatments include polysiloxane treatments such as those disclosed in U.S. Pat. No. 5,143,722.

### 8. UV Actives (Light Protective Filters)

The compositions of the instant disclosure may contain a UV active. As used herein, a “UV active” or a “Light Protective Filter” includes both sunscreen agents and physical sunblocks. Suitable UV actives may be organic or inorganic. Suitable UV actives are listed in the functional category of “Sunscreen Agents” in the Personal Care Product Council's International Cosmetic Ingredient Dictionary and Handbook, Thirteenth Edition, 2010.

Suitable UV actives include dibenzoylmethane derivatives including 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropyldibenzoylmethane, 4,4′ dimethoxy dibenzoylmethane, 4-tert-butyl-4′-methoxy dibenzoylmethane (i.e., butyl methoxydibenzoylmethane or avobenzone) (commercially available as PARSOL® 1789 from DSM), 2-methyl-5-isopropyl-4′-methoxy dibenzoylmethane, 2-methyl-5-tert-butyl-4′-methoxydibenzoylmethane, 2,4-dimethyl-4′-methoxy dibenzoylmethane, and 2,6-dimethyl-4-tert-butyl-4′-methoxy dibenzoylmethane. Other suitable UV actives include 2-ethylhexyl-p-methoxycinnamate (commercially available as PARSOL® MCX from DSM), 2-hydroxy-4, methoxybenzophenone, benzonphenone-3 (i.e., oxybenone), octyldimethyl-p-aminobenzoic acid, digalloyltrioleate, 2,2-dihydroxy-4-methoxybenzophenone, ethyl-4-(bis(hydroxy-propyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene, zinc oxide, titanium dioxide, and mixtures of these compounds.

Particularly suitable UV actives useful in the compositions of the present disclosure are 2-ethylhexyl-p-methoxycinnamate, 4-tert-butyl-4′-methoxy dibenzoylmethane, 2-hydroxy-4-methoxybenzo phenone, 2-phenylbenzimidazole-5-sulfonic acid, octocrylene, zinc oxide, titanium dioxide, and mixtures thereof.

Other suitable UV actives include 4-methylbenzylidene camphor (commercially available as PARSOL® 5000 from DSM or Eusolex 6300 from Merck), methylene bis-benzotriazolyl tetramethylbutylphenol (i.e., bisoctrizole, commercially available as Tinosorb® M from BASF), bis-ethylhexyloxyphenol methoxyphenol triazine (i.e., bemotrizinol, commercially available as Tinosorb® S from BASF), disodium phenyl dibenzimidazole tetrasulfonate (i.e., Bisdisulizole disodium, commercially available as Neo Heliopan® AP from Symrise), Ethylhexyl triazone (commercially available as Uvinul® T 150 from BASF), Drometrizole trisiloxane (marketed as Mexoryl XL by L'Oreal), Sodium Dihydroxy Dimethoxy Disulfobenzophenone (i.e., benzophenone-9, commercially available as Uvinul® DS 49 from BASF), Diethylamino Hydroxybenzoyl Hexyl Benzoate (commercially available as Uvinul® A Plus from BASF), diethylhexyl butamido triazone (i.e., Iscotrizinol, commercially available as Uvasorb® HEB by 3V Sigma), Polysilicone-15 (i.e., commercially available as PARSOL® SLX from DSM), and Isoamyl p-Methoxycinnamate (i.e., amiloxate, commercially available as Neo Heliopan® E 1000 from Symrise).

### 9. Photostabilizers

A suitable photostabilizer is alpha-cyanodiphenylacrylate is as disclosed in U.S. Pat. No. 7,713,519. The alpha-cyanodiphenylacrylate may have the general formula:

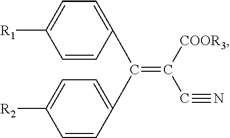

wherein one or both of R1 and R2 is independently a straight or branched chain C1-30 alkoxy radical and any non-alkoxy R1 or R2 radical is hydrogen; and R3 is a straight or branched chain C1-30 alkyl. Alternately, one or both of R1 and R2 is independently a C1-8 alkoxy radical and any non-alkoxy R1 or R2 radical is hydrogen; and R3 is a straight of branched chain C2-20 alkyl. Alternately, one or both of R1 and R2 is independently methoxy, and any non-methoxy R1 or R2 is hydrogen; and R3 is a straight or branched chain C2-20 alkyl.

A suitable alpha-cyanodiphenylacrylate is ethylhexyl methoxycrylene, or 2-ethylhexyl 2-cyano-3-(4-methoxyphenyl)-3-phenylpropenoate, wherein R1 is methoxy, R2 is hydrogen, and R3 is 2-ethylhexyl. This material is available from Hallstar Company under trade name Solastay® S 1.

Another suitable photostabilizer includes diesters or polyesters of naphthalene dicarboxylic acid as disclosed in U.S. Pat. Nos. 5,993,789, 6,113,931, 6,126,925 and 6,284,916. Suitable diesters or polyesters of naphthalene dicarboxylic acid may have the following formula:

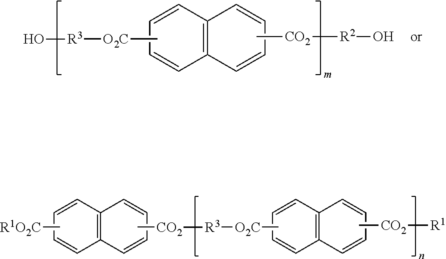

wherein each R¹ independently is an alkyl group having 1 to 22 carbon atoms, or a diol having the formula HO—R²—OH, or a polyglycol having the formula HO—R³—(—O—R²—)ₘ—OH, and, wherein R² and R³, same or different, are each an alkylene group, straight chain or branched, having 1 to 6 carbon atoms, wherein m and n are each 1 to about 100, 1 to about 10, or 2 to about 7. A suitable diesters of naphthalene dicarboxylic acid is diethylhexyl 2,6-naphthalate available as Corapan® TQ from Symrise.

Another suitable photostabilizer is 4-hydroxybenzylidenemalonate derivatives or 4-hydroxycinnamate derivatives. Suitable materials may have the following formula:

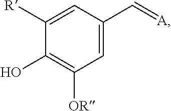

wherein A is a chromophoric group that absorbs UV-radiation, comprises one divalent group or two monovalent groups with at least one group having carbonyl (C═O) functionality; R′ is hydrogen, a linear or branched C₁-C₈ alkyl radical or a linear or branched C₁-C₈ alkoxy radical; and R″ is a linear or branched C₁-C₈ alkyl radical. Exemplary compounds include ethyl-alpha-cyano-3,5-dimethoxy-4-hydroxy cinnamate, ethyl-alpha-acetyl-3,5-dimethoxy-4-hydroxy cinnamate, iso-propyl-alpha-acetyl-3,5-dimethoxy-4-hydroxy cinnamate, iso-amyl-alpha-acetyl-3,5-dimethoxy-4-hydroxy cinnamate, 2-ethylhexyl-alpha-acetyl-3,5-dimethoxy-4-hydroxy cinnamate, diethyl-3,5-dimethoxy-4-hydroxy benzylidene malonate, di-(2-ethylhexyl)-3,5-dimethoxy-4-hydroxy benzylidene malonate, diisoamyl-3,5-dimethoxy-4-hydroxy benzylidene malonate, didodecyl-3,5-dimethoxy-4-hydroxy benzylidene malonate, dipalmitoyl-3,5-dimethoxy-4-hydroxy benzylidene malonate, and di-isopropyl-3,5-dimethoxy-4-hydroxy benzylidene malonate. A particularly suitable compound is diethylhexyl syringylidenemalonate (INCI name) available under the tradename Oxynex® ST from EMD Chemicals, Inc., having the formula:

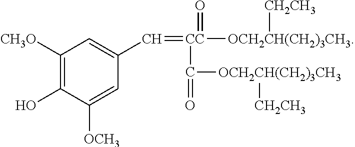

Additional suitable 4-hydroxybenzylidenemalonate derivatives or 4-hydroxycinnamate derivatives are disclosed in U.S. Pat. No. 7,357,919 and U.S. Patent Application Publication No. 2003/0108492A1 and US2003/0157035A.

Another suitable photostabilizer is a 2-pyrrolidinone-4-carboxy ester compounds. Suitable 2-pyrrolidinone-4-carboxy ester compounds may have the following formula:

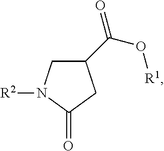

wherein R¹ is a linear or branched C₁-C₂₀ alkyl radical, and R² is a linear or branched C₁-C₂₀ alkyl radical which can contain a C₅-C₆ ring, the phenyl radical, the benzyl radical or the phenethyl radical. Exemplary radicals for R¹ and R² include methyl, ethyl, n-propyl, isopropyl, n-butyl, isobutyl, tert-butyl, n-octyl, 2-ethylhexyl, dodecyl, hexadecyl, cyclohexyl and methylcyclohexyl radicals. Particular examples of 2-pyrrolidinone-4-carboxy ester compounds are provided in U.S. Patent Application Publication No. 2010/0183529.

Other suitable photostabilizers include: silicon-containing s-triazines substituted with two aminobenzoate or aminobenzamide groups as described in U.S. Patent Application Publication No. 2008/0145324; fluorene derivatives as described in U.S. Patent Application Publications Nos. 2004/00579912, 2004/00579914, 200/00579916, and 2004/062726; piperidinol salts as described in U.S. Patent Application Publications No. 2005/0220727 including tris(tetramethylhydroxypiperidinol) citrate sold under the tradename Tinogard® Q by Ciba; and arylalkyl amides and esters as described in U.S. Patent Application Publication No. 2008/0019930.

Other suitable photostabilizers are listed in the functional category of “Light Stabilizers” in the Personal Care Product Council's International Cosmetic Ingredient Dictionary and Handbook, Thirteenth Edition, 2010.

In one embodiment, the personal care composition may comprise from about 0.0001%, 0.001%, 0.01%, 0.05%, 0.1%, 0.5%, or 1% to about 30%, 25% 20%, 10%, 7%, or 5%, by weight of the composition, of one or more suitable photostabilizer. In certain embodiments, the personal care composition may comprise at least one photostabilzer and at least one UV active. In particular embodiments, the UV active is a dibenzoylmethane derivative. In a particular embodiment, the UV active is 4,4′-t-butyl methoxydibenzoyl-methane (i.e., avobenzone).

### 10. Anti-Oxidants/Racial Scavengers

The personal care compositions of the present disclosure can include an anti-oxidant/radical scavenger. In one embodiment, the composition may comprise from about 0.0001%, 0.001%, 0.01%, 0.05%, 0.1%, 0.5%, or 1% to about 30%, 25%, 20%, 10%, 7%, 5%, or 3%, by weight of the composition, of one or more anti-oxidant/radical scavengers.

Suitable anti-oxidants are listed in the functional category of “Antioxidants” in the Personal Care Product Council's International Cosmetic Ingredient Dictionary and Handbook, Thirteenth Edition, 2010.

Suitable anti-oxidants include butylated hydroxytoluene (BHT) and butylated hydroxyanisole (BHA). BHT can be described by the general formula:

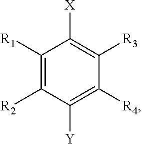

wherein X is OH or SH;
Y is selected from the group consisting of H, OH, OR₅, COOR₅, alkyl, cycloalkyl, heteroalkyl, heterocycloalkyl, aromatic, heteroaromatic, carboxamido, sulfonamido, carbamate, urea, and trialkylsilyl;
R₁, R₂, R₃, R₄ are selected from the group consisting of alkyl, cycloalkyl, heteroalkyl, heterocycloalkyl, aromatic, heteroaromatic, OR₅, carboxamido, sulfonamido, formyl, acyl, carboxyl, carboxylate, carbamate, urea, trialkylsilyl, hydroxyl, and hydrogen;
R₅ is selected from the group consisting of alkyl, cycloalkyl, heteroalkyl, heterocycloalkyl, aromatic, heteroaromatic, trialkylsilyl, acyl, and hydrogen.

Other anti-oxidants/radical scavengers such as ascorbic acid (vitamin C), tocopherol (vitamin E), tocopherol sorbate, tocopherol acetate, other esters of tocopherol, 6-hydroxy-2,5,7,8-tetramethylchroman-2-carboxylic acid (commercially available under the tradename Trolox®), amines (e.g., N,N-diethylhydroxylamine, amino-guanidine), nordihydroguaiaretic acid, bioflavonoids, amino acids, silymarin, sorbic acids and its salts, lipoic acid, olive extracts, green tea extracts, white tea extracts, black tea extracts, polyphenols such as proanthocyanidine from pine bark, carotenoids, curcumin compounds such as tetrahydrocurcumin, OCTA (L-2-oxo-4-thiazolidine carboxylic acid), glutathione, and grape skin/seed extracts may be used. Suitable anti-oxidants/radical scavengers can be selected from esters of tocopherol such as tocopherol acetate.

In one embodiment, the personal care composition comprises tocopherol sorbate. As used herein, “tocopherol sorbate” refers to the sorbic acid ester of tocopherol, a detailed description of which can be found in issued U.S. Pat. No. 5,922,758. In one embodiment, the composition may comprise from about 0.0001%, 0.001%, 0.01%, 0.05%, 0.1%, 0.5%, or 1% to about 50%, 25%, 20%, 10%, 7%, or 5%, by weight of the composition, of the tocopherol sorbate.

### 11. Botanical Extracts

The personal care compositions of the instant disclosure may comprise botanical extracts. In one embodiment, the composition may comprises from about 0.0001%. 0.0005% 0.001%, 0.01%, 0.05%, 0.1%, 0.5%, or 1% to about 30%, 25%, 20%, 10%, 7%, 5%, 3%, by weight of the composition, of one or more botanical extracts. Suitable botanical extracts include extracts from plants (herbs, roots, flowers, fruits, seeds) such as flowers, fruits, vegetables, and so on, including yeast ferment extract, Padina Pavonica extract, thermus thermophilis ferment extract, camelina sativa seed oil, Boswellia serrata extract, olive extract, bodopsis Thaliana extract, Acacia Dealbata extract, Acer Saccharinim (sugar maple), acidopholus, acorus, aesculus, Alicaligenes polysaccharides, agaricus, agave, agrimonia, algae, aloe, citrus, brassica, cinnamon, orange, apple, blueberry, cranberry, peach, pear, lemon, lime, pea, seaweed, caffeine, green tea, chamomile, willowbark, mulberry, poppy, and the like. Further specific examples include, but are not limited to, Glycyrrhiza Glabra, Salix Nigra, Macrocyestis Pyrifera, Pyrus Malus, Saxifraga sarmentosa, Vitis vinifera, Morus nigra, Scutellaria baicalensis, Anthemis nobilis, Salvia sclarea, Rosmarinus officianalis, Citrus medica limonum, Ginkgo biloba Panax ginseng, Siegesbeckia Orientalis, Fructus Mume, Ascophyllum Nodosum, Bifida Ferment lysate. Glycine Soja extract, Beta Vulgaris, Haberlea rhodopensis, Polygonum cuspidatum, Citrus Aurantium dulcis, Vitis vinifera, Selaginella tamariscina, Humulus lupulus, Citrus reticulata Peel, Punica granatum, Asparagopsis, Curcuma longa, Menyanthes trifoliata, Helianthus annuus, Hordeum vulgare, Cucumis sativus, Evernia prunastri, Evernia furfuracea, Laminaria angustata, Laminaria cloustoni, Laminaria digitata, Laminaria digitata, Laminaria hyperborea, Laminaria japonica, Laminaria longissima, Laminaria ochotensis, Laminaria ochroleuca, Laminaria saccharina, and mixtures thereof. Other suitable actives are listed in the functional category of “Biological Products” in the Personal Care Product Council's International Cosmetic Ingredient Dictionary and Handbook, Thirteenth Edition, 2010.

Any other suitable optional component can also be included in the personal care composition of the present disclosure, such as those ingredients that are conventionally used in given product types. The Personal Care Product Council's International Cosmetic Ingredient Dictionary and Handbook, Thirteenth Edition, 2010, describes a wide variety of nonlimiting functional materials that can be added to the composition herein. Examples of these functional classes include, but are not limited to: abrasives, absorbents, fragrances, anti-acne agents, anti-caking agents, antifoaming agents, antimicrobial agents (e.g., iodopropyl butylcarbamate), antifungal agents, antioxidants, binders, buffering agents, bulking agents, chelating agents, colorants, cosmetic astringents, cosmetic biocides, denaturants, drug astringents, external analgesics, film formers, opacifying agents, pH adjusters, plant derivatives, plant extracts, plant tissue extracts, plant seed extracts, plant oils, botanicals, botanical extracts, preservatives, propellants, reducing agents, sebum control agents, sequestrants, skin bleaching agents, skin-conditioning agents (e.g. humectants and occlusive agents), and skin protectants. Other suitable optional person care ingredients include materials listed in paragraphs 513-839 of U.S. Patent Application No. 2010/0112100.

### Methods for Preventing or Reducing Hyperpigmentation

Another aspect of the disclosure is directed to a method for preventing or reducing hyperpigmentation in a subject, comprising administering the subject a personal care composition described herein. As used herein, the subject includes a human.

Another aspect of the disclosure is directed to a method for preventing or reducing hyperpigmentation in a subject, comprising administering the subject a personal care composition comprising a skin lightening mixture, and an antioxidant.

In some embodiments, the skin lightening mixture comprises niacinamide, extract of sea daffodil (Pancratium maritimum), and acetyl Glycyl β-Alanine.

In some embodiments, the skin lightening mixture comprises by weight: (a) 0.1%-10% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) niacinamide, (b) 0.1%-10% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) extract of sea daffodil (Pancratium maritimum), (c) 0.1%-10% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%) acetyl Glycyl β-Alanine.

In some embodiments, the antioxidant is a Vitamin C derivative.

In some embodiments, the Vitamin C derivative is ascorbyl palmitate having the formula:

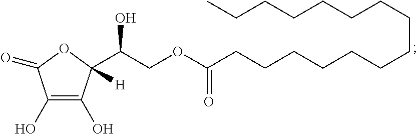 [SMILES: CCCCCCCCCCCCCCCC(=O)OC[C@H](O)[C@H]1OC(=O)C(O)=C1O]

derivatives thereof; and salts of any of the foregoing.

In some embodiments, the vitamin C derivative is magnesium ascorbyl phosphate having the formula:

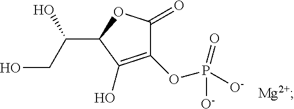 [SMILES: O=C1O[C@H]([C@@H](O)CO)C(O)=C1OP(=O)([O-])[O-]]

derivatives thereof; and salts of any of the foregoing.

In some embodiments, the vitamin C derivative is ethyl ascorbic acid having the formula:

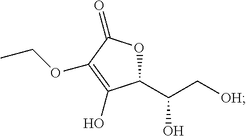

derivatives thereof; and salts of any of the foregoing.

In some embodiments, the vitamin C derivative is ascorbyl glucoside (AA-2G) having the formula:

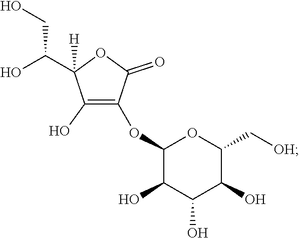 [SMILES: O=C1O[C@H]([C@H](O)CO)C(O)=C1O[C@H]1O[C@H](CO)[C@@H](O)[C@H](O)[C@H]1O]

derivatives thereof; and salts of any of the foregoing.

In some embodiments, the composition comprises 0.1%-5% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%) Vitamin C derivative. In some embodiments, the composition comprises 0.1%-2% (e.g., 0.1%-10% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%) Vitamin C derivative by weight. In some embodiments, the composition comprises 0.1%-1% (e.g., 0.1%, 0.2%, 0.3%, 0.4%, 0.5%, 0.6%, 0.7%, 0.8%, 0.9%, 1%) Vitamin C derivative by weight. In some embodiments, the composition comprises 0.5%-1% % (e.g., 0.5%, 0.6%, 0.7%, 0.8%, 0.9%, 1%) Vitamin C derivative by weight. In some embodiments, the composition comprises 1% Vitamin C derivative by weight.

Another aspect of the disclosure is directed to a method for preventing or reducing hyperpigmentation in a subject, comprising administering the subject a personal care composition comprising, by weight: (a) 0.1%-10% niacinamide, (b) 0.1%-10% extract of sea daffodil (Pancratium maritimum), (c) 0.1%-10% acetyl Glycyl β-Alanine, and (d) 0.1%-5% Vitamin C derivative.

In some embodiments, the personal care composition further comprises at least one of the following: water, humectant, pigment, antioxidant, preservative, gelling agent, emulsifier, solvent, pH balancing agent, and emollient.

In some embodiments, the humectant comprises at least one of xylitylglucoside, anhydroxylitol, and xylitol.

In some embodiments, the pigment comprises at least one of zinc oxide, titanium dioxide and iron oxide.

In some embodiments, the personal care composition further comprises at least one of the following: hydrolyzed hyaluronic acid, glycerin, sodium hydroxide, phenoxyethanol, xantham gum, sodium polyacryloyldimethyl taurate, undecane, tridecane, tocopherol, dimethicone, titanium dioxide, tin oxide, and calcium aluminum borosilicate.

In some embodiments, the personal care composition comprises at least one of the following: hydrolyzed hyaluronic acid (0.001%-0.05% by weight of the personal care composition), glycerin (0.1%-7% by weight), sodium hydroxide (0.001%-0.1% by weight of the personal care composition), phenoxyethanol (0.1%-1% by weight of the personal care composition), xantham gum (0.05%-0.5% by weight of the personal care composition), sodium polyacryloyldimethyl taurate (0.1%-1.2% by weight of the personal care composition), an emollient composition comprising undecane, tridecane, and tocopherol (0.05%-0.5% by weight of the personal care composition), dimethicone (0.5%-5% by weight), a pigment composition comprising titanium dioxide, tin oxide, and calcium aluminum borosilicate (0.005%-0.05% by weight of the personal care composition).

In some embodiments, the emollient composition comprises Cetiol Ultimate™ from BASF which comprises undecane, tridecane, and tocopherol. In some embodiments, the personal care composition comprises between 0.05% and 0.5% (e.g., 0.05%, 0.06%, 0.07%, 0.08%, 0.09%, 0.1%, 0.2%, 0.3%, 0.4%, or 0.5%) Cetiol Ultimate™.

In some embodiments, the pigment composition comprises Syncrystal Gold™ from Eckart which comprises titanium dioxide, tin oxide, and calcium aluminum borosilicate. In some embodiments, the personal care composition comprises between 0.005% and 0.05% (e.g., 0.005%, 0.01%, 0.02%, 0.03%, 0.04%, or 0.05%) Syncrystal Gold™.

In some embodiments, the personal care composition further comprises an exfoliator. In some embodiments, the exfoliator is jojoba beads containing vitamin E.

In some embodiments, the personal care composition is a lotion, a gel, a mask, or a spray.

In some embodiments, the personal care composition is administered topically. In some embodiments, the personal care composition is administered every 2 hours, 3 hours, 6 hours, 12 hours, or every day, every other day, or every three days. In some embodiments, the personal care composition is administered for at least 10, 11, 12, 13, 14, 15, 16, 17, 18, 19 or 20 days before a reduction in hyperpigmentation or dark spots is observed.

In some embodiments, the subject suffers from sensitive skin.

The disclosure now being generally described, it will be more readily understood by reference to the following example which is included merely for purposes of illustration of certain aspects and embodiments of the present disclosure, and are not intended to limit the disclosure.

### EXAMPLES

### Example 1: Materials and Methods

### Cellular Models

Normal Human Dermal Fibroblasts—NHDFs. Normal Human foreskin-derived Dermal Fibroblasts (NHDFs; ATCC, CRL-2522) cells were grown in Dulbecco's Modified Eagle Medium (DMEM, Gibco/Life Technologies, 31885) supplemented with 10% of Foetal Bovine Serum (FBS, Gibco/Life Technologies, 10270) and antibiotics (Penicillin/Streptomycin, Gibco/Life Technologies, 15140). The cells were maintained in a humidified incubator at 37° C. with a 5% CO₂ atmosphere.

Normal Human Epidermal Keratinocytes—NHEKs. The Normal Human foreskin-derived Epidermal Keratinocytes (NHEKs, Lonza, 00192906) were grown in Epilife medium (Fisher Scientific, M-EPI-500-A) supplemented with Human Keratinocyte Growth Supplement (HKGS, Fisher Scientific, S-001-5) and antibiotics (Gentamycin, Fisher Scientific, 15710-049). The cells were maintained in a humidified incubator at 37° C. with a 5% CO₂ atmosphere.

Normal Human Epidermal Melanocytes—NHEMs-DP. Normal Human Epidermal Melanocytes darkly-pigmented (NHEM-DP; Thermo Fisher Scientific, C1025C) were grown in M254 medium (Thermo Fisher Scientific, M254500) supplemented with Human Melanocyte Growth Supplement (HMGS; Thermo Fisher Scientific, S0025) and antibiotics (Gentamycin, Thermo Fisher Scientific, 15710049). The cells were maintained in a humidified incubator at 37° C. with a 5% CO₂ atmosphere.

### Dose Determination Experiments

In order to determine the optimal concentrations of the formulations to apply in specific cellular models of the study, preliminary dose-finding studies were carried out on NHDFs and on NHEKs and NHEMs, such as described after, on basal conditions.

For NHDFs fibroblasts, after amplification, cells were plated in 24-well plates containing complete medium (containing Foetal Bovine Serum; FBS). Then, NHDFs fibroblasts were treated with the formulations placed, without filtration, in the culture medium deprived in FBS for the next 24 h.

For NHEKs keratinocytes, after amplification, cells were plated in 24-well plates containing complete medium. Then, NHEKs keratinocytes were treated with the 6 formulations placed, without filtration, in the culture medium for the next 24 h.

For NHEMs melanocytes, after amplification, cells were plated in 24-well plates containing complete medium. Then, NHEMs melanocytes were treated with the 6 formulations placed, without filtration, in the culture medium for the next 96 h, with a medium refresh after the first 48 h.

The effects of the treatments were compared to the untreated control condition and, to a positive cytotoxicity reference (SDS-Sodium Dodecyl Sulfate). All the treatments were performed in triplicates (n=3).

At the end of the treatment with 5 concentrations of formulations, the cell viability was evaluated using the MTS (3-(4,5-dimethythiazol-2-yl)-5-(3-carboxymethoxy-phenyl)-2-(4-sulfophenyl)-2H-tetrazolium) assay.

### Antioxidant (ROS Production) Assays

The evaluation of the intracellular reactive oxygen species (ROS) production induced by UVA irradiation was performed using the H2DCFDA (2′-7′dichlorodihydrofluorescein diacetate; Fisher Scientific, D399) probe. The H2DCFDA is a dye which, after diffusing into the cells and its acetate groups cleaved by intracellular esterases, can be oxidized by free radicals in DCF (2′-7′ dichlorofluorescein), a fluorescent compound. Production of ROS is measured through the fluorescence level emitted by the oxidized dye.

For this assay, the NHEKs keratinocytes have been seeded in 24-well plates 24 h before to be treated, in the culture medium, with reference and formulations in triplicates (n=3), for the next 24 h. At the end of treatment, cells were rinsed with PBS and the probe was loaded into the cells by an incubation of 30 min (15 min at the concentration of 10 μM and 15 min at 5 μM) in PBS without test or reference molecules, at 37° C. and 5% CO₂ atmosphere. Afterwards, cultures were rinsed with PBS and placed into fresh PBS (with test formulations and reference compound) for irradiation to UVA (5 J/cm²; ˜20 min) using a Biosun (Vilber Lourmat, FR). Unirradiated cells placed in PBS for 20 min were used as reference condition. After the UVA challenge, the fluorescence was measured (GloMax Explorer GM3500, Microplate Reader).

After ROS measurement, the cell metabolic activity (MTS assay) was also evaluated from the same cell cultures and the obtained fluorescence intensities were normalized to MTS data.

The effects of the treatments were compared to the untreated control condition. In parallel to cell treatment with the test formulations, L (+)-ascorbic acid (vitamin C; VWR, 83568.180) was used as antioxidant reference compound for validation of the assay. The reference was applied at 300 μg/ml on cell cultures according to the same experimental scheme.

### Melanin Content Assays

At the end of treatment, NHEM-DP cells (n=3) were lysed using NaOH (1N; Merck, 109956) solution. From the collected extracts, the protein content was quantified by a colorimetric assay (Pierce Coomassie Protein Assay Kit, Thermo Fischer Scientific, 23200) using a spectrophotometer (GloMax Explorer GM3500, Microplate Reader) for reading of the optical density at 600 nm. The melanin content was determined after sample heating for 1 h at 80° C. for its solubilization and reading of the optical density at 490 nm. Afterwards, the amount of melanin was determined on the basis of a standard curve using synthetic melanin (Sigma Aldrich, 8631) and then normalized to the total protein content.

### Tyrosinase Activity Assays

At the end of the treatment, the NHEMs-DP melanocytes were lysed using a specific buffer composed of 0.1 M potassium/phosphate solution at pH 6.8 and 0.5% triton. The cell lysates were placed in an ultrasonic bath, then centrifuged in order to harvest the protein fraction. The protein concentration was determined by a colorimetric assay based on detection with bicinchoninic acid (Pierce BCA Protein Assay Kit; Thermo Fisher Scientific, 23227) and an optical density reading at a wavelength of 562 nm by spectrophotometry. The tyrosinase activity was then evaluated by incubation, for 2 h at 37° C., of an equivalent protein quantity of each sample with the substrate of the enzyme (L-DOPA or 3,4-Dihydroxy-L-phenylalanine; 10 mM), and reading the optical density at a wavelength of 490 nm.

### Example 2: Antioxidant Assay

Production of reactive oxygen species (ROS) was measured on ultraviolet A (UVA)-treated normal human epidermal keratinocytes which were pre-treated for 24 hours with test products having differing amounts of Vitamin C derivatives. The test products were diluted to a concentration of 0.04%. The experiments were carried out in triplicate.

Test products:
Vitamin C Lotion 15% (comprises 15% Tetrahexyldecyl Ascorbate)
Complex 30% (comprises 30% Tetrahexyldecyl Ascorbate)
L-Ascorbic Acid solution (comprises 15% L-Ascorbic Acid)
Test Serum (comprises, by weight: (a) 3%-6% niacinamide, (b) 0.5%-3% extract of sea daffodil (Pancratium maritimum), (c) 0.5%-3% acetyl glycyl β-alanine, and (d) 1% ethyl ascorbic acid)
Vitamin C Serum 1 (comprises 10% ascorbic acid)
Vitamin C Serum 2 (comprises 10% L-ascorbic acid)

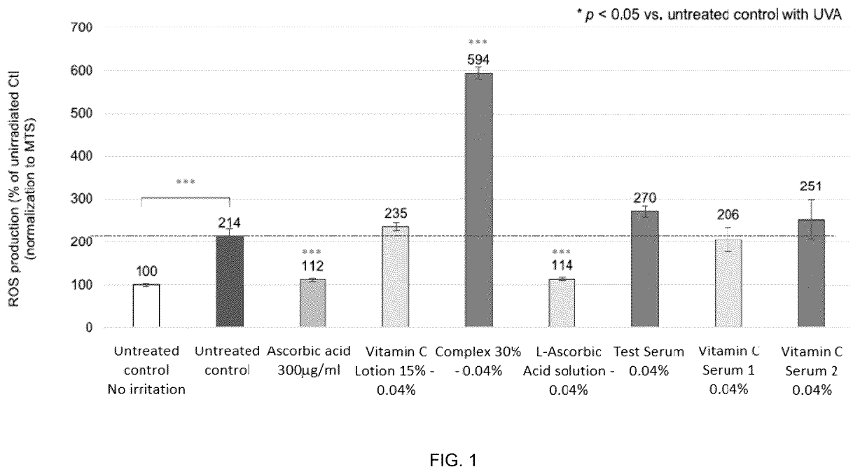

FIG. 1 shows the result of the ROS experiments. Exposure of human epidermal keratinocytes to UVA radiation induced significantly the ROS production (2.1× increase), as compared to unirradiated cells. L-Ascorbic Acid solution (15% Vitamin C derivative) demonstrated the best antioxidant effect among all formulations. Complex 30% (30% Vitamin C derivative) was toxic for cells, so no result was gathered in this assay. The test Serum with 1% Vitamin C derivative surprisingly performed as well as Vitamin C Lotion 15% (15% Vitamin C derivative), Vitamin C Serum 1 (10% Vitamin C derivative) and Vitamin C Serum 2 (10% Vitamin C derivative).

### Example 3: Skin Brightening Assays

Normal human epidermal keratinocytes were treated for 72 hours with each test product (0.004% concentration for the melanin reduction assay, 0.04% concentration for the tyrosinase assay). Tyrosinase activity was measured by optical density reading with the enzyme substrate. The experiments were carried out in triplicate.

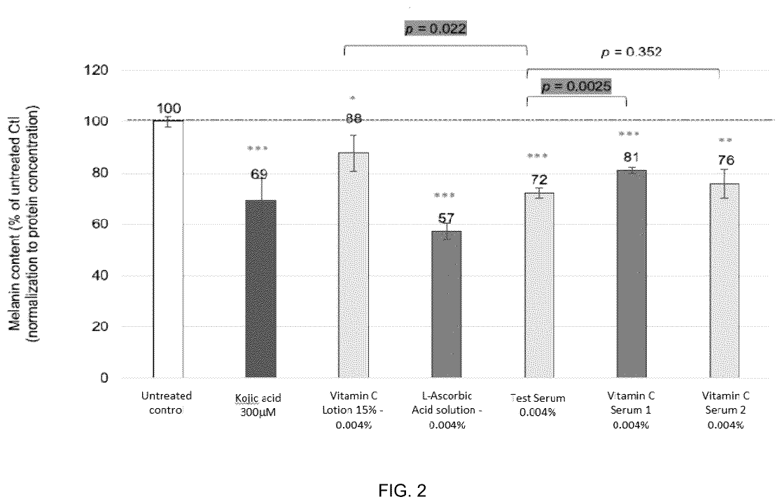

At the end of the treatments, intracellular melanin was extracted from cell cultures and analyzed as described in materials and methods in Example 1. The analysis of Complex 30% was not possible, given the cytotoxicity detected in the preliminary experiment in this model, for this condition. FIG. 2 shows the results of melanin reduction assays. Kojic acid was used as reference of whitening condition which significantly decreased the melanin content within darkly pigmented melanocytes, validating the assay system and the analysis method. Interestingly, when compared to the untreated condition, the cell treatment with each test formulations significantly reduced the melanin content, with greater effect for L-Ascorbic Acid solution and then Test serum, Vitamin C serum 2 and Vitamin C Serum 1 at 0.004%. Indeed, the level of intracellular melanin amount decreased remarkably by around 2× in response to L-Ascorbic Acid solution treatment, supporting its significant and interesting influence on melanogenesis.

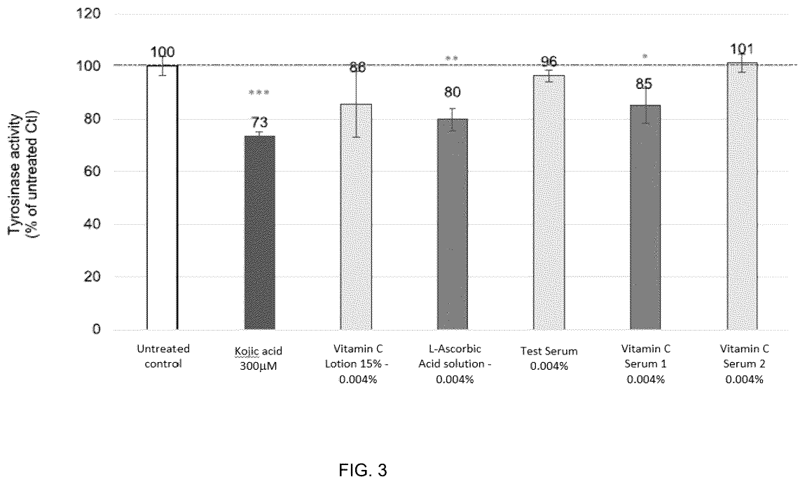

FIG. 3 shows the results of tyrosinase inhibition assays. The treatments with the formulations revealed significant reduction of tyrosinase activity for L-Ascorbic Acid solution and for Vitamin C Serum 1. This is concomitant with a melanin content reduction already observed for these conditions. The 20% reduction of enzyme activity by the L-Ascorbic Acid solution treatment thus revealed that other mechanisms are probably involved to explain the stronger melanin content reduction in FIG. 3. Moreover, although not significant, the downward trend observed for the Vitamin C lotion condition also confirmed that the reduction in melanin content was directly related to the slight decrease of tyrosinase activity.

The absence of effect of Test serum and Vitamin C Serum 2 on tyrosinase activity means that reduction of melanin content detected by treatment of melanocytes with these compounds was the consequence of other mode of action.

1. A topical personal care composition comprising, by weight:
(a) 0.1%-10% niacinamide;
(b) 0.1%-10% extract of sea daffodil (Pancratium maritimum);
(c) 0.1%-10% acetyl glycyl β-alanine; and
(d) 0.1%-5% vitamin C derivative.

2. The topical personal care composition of claim 1, wherein the Vitamin C derivative is selected from ethyl ascorbic acid, ascorbyl glucoside (AA2G), ascorbic acid, ascorbyl palmitate, or magnesium ascorbyl phosphate (MAP).

3. The topical personal care composition of claim 1, wherein the topical personal care composition comprises 0.1%-2% vitamin C derivative by weight, relative to the total weight of the topical personal care composition.

4. The topical personal care composition of claim 1, wherein the topical personal care composition comprises 0.1%-1% Vitamin C derivative by weight, relative to the total weight of the topical personal care composition.

5. The topical personal care composition of claim 1, further comprising at least one of the following: water, a humectant, a pigment, an antioxidant, a preservative, a gelling agent, an emulsifier, a solvent, a pH balancing agent, and an emollient.

6. The topical personal care composition of claim 5, wherein the humectant comprises at least one of xylitylglucoside, anhydroxylitol, and xylitol.

7. The topical personal care composition of claim 5, wherein the pigment comprises at least one of zinc oxide, titanium dioxide, and iron oxide.

8. The topical personal care composition of claim 1, further comprising at least one of the following: hydrolyzed hyaluronic acid, glycerin, sodium hydroxide, phenoxyethanol, xanthan gum, sodium polyacryloyldimethyl taurate, undecane, tridecane, tocopherol, dimethicone, titanium dioxide, tin oxide, and calcium aluminum borosilicate.

9. The topical personal care composition of claim 1, wherein the topical personal care composition is a lotion, a gel, a mask, or a spray.

10. A method for preventing or reducing hyperpigmentation in the skin of a subject, the method comprising:
administering to the skin of a subject a topical personal care composition comprising:
a skin lightening mixture comprising niacinamide, extract of sea daffodil (Pancratium maritimum), and acetyl Glycyl β-Alanine; and
an antioxidant comprising a vitamin C derivative.

11. The method of claim 10, wherein the topical personal care composition comprises, by weight:
(a) 0.1%-10% niacinamide;
(b) 0.1%-10% extract of sea daffodil (Pancratium maritimum);
(c) 0.1%-10% acetyl Glycyl β-Alanine; and
(d) 0.1%-5% vitamin C derivative.

12. The method of claim 10, wherein the Vitamin C derivative is selected from ethyl ascorbic acid, ascorbyl glucoside (AA2G), ascorbic acid, ascorbyl palmitate or magnesium ascorbyl phosphate (MAP).

13. The method of claim 10, wherein the topical personal care composition comprises 0.1%-2% vitamin C derivative by weight, relative to the total weight of the topical personal care composition.

14. The method of claim 10, wherein the topical personal care composition comprises 0.1%-1% vitamin C derivative by weight, relative to the total weight of the topical personal care composition.

15. The method of claim 10, wherein the topical personal care composition further comprises at least one of the following: water, a humectant, a pigment, an antioxidant, a preservative, a gelling agent, an emulsifier, a solvent, a pH balancing agent, and an emollient.

16. The method of claim 15, wherein the humectant comprises at least one of xylitylglucoside, anhydroxylitol, and xylitol.

17. The method of claim 15, wherein the pigment comprises at least one of zinc oxide, titanium dioxide, and iron oxide.

18. The method of claim 10, wherein the topical personal care composition further comprises at least one of the following: hydrolyzed hyaluronic acid, glycerin, sodium hydroxide, phenoxyethanol, xanthan gum, sodium polyacryloyldimethyl taurate, undecane, tridecane, tocopherol, dimethicone, titanium dioxide, tin oxide, and calcium aluminum borosilicate.

19. The method of claim 10, wherein the topical personal care composition is a lotion, a gel, a mask, or a spray.

20. The method of claim 10, wherein the subject suffers from sensitive skin.
